(19) United States
(12) Patent Application Publication
(10) Pub. No.: US 2009/0028797 A1
(43) Pub. Date: Jan. 29, 2009

(54) Novel polymeric ultrasound contrast agent and methods of making thereof

(75) Inventors: Margaret A. Wheatley, Media, PA (US); John Lewandowski, Coopersburg, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Drexel University

(21) Appl. No.: 12/157,755

(22) Filed: Jun. 13, 2008

Related U.S. Application Data
(60) Provisional application No. 60/944,026, filed on Jun. 14, 2007.

(51) Int. Cl. A61K 49/22

(52) U.S. Cl. 424/9.5

(57) ABSTRACT

The present invention provides a novel method of manufacturing nanosized polymeric echogenic contrast agents. The method of the present invention comprises a modified salting out process which results on nanosized polymeric capsules encapsulating an aqueous core that is subsequently evacuated. The compositions of the present invention can be used as contrast agents as well as to deliver therapeutic agents to specific targets.

### CROSS REFERENCE TO RELATED APPLICATIONS

Application claims priority under 35 U.S.C. § 119(e) to U.S. Provisional Application No. 60/944,026, filed on Jun. 14, 2007, which application is incorporated by reference herein in its entirety.

### STATEMENT REGARDING FEDERALLY SPONSORED RESEARCH OR DEVELOPMENT

This invention was made, in part, using fund obtained from the U.S. Government (National Institutes of Health Grant No. CA102238), and the U.S. Government therefore has certain rights in this invention.

### BACKGROUND OF THE INVENTION

Ultrasound contrast agents are used routinely in medical diagnostic, as well as industrial, ultrasound. For medical diagnostic purposes, contrast agents are usually gas bubbles, which derive their contrast properties from the large acoustic impedance mismatch between blood and the gas contained therein. Important parameters for the contrast agent include particle size, imaging frequency, density, compressibility, particle behavior (surface tension, internal pressure, bubble-like qualities), and biodistribution and tolerance.

Gas-filled particles are by far the best reflectors. Various bubble-based suspensions with diameters in the 1 to 15 micron range have been developed for use as ultrasound contrast agents. Bubbles of these dimensions have resonance frequencies in the diagnostic ultrasonic range, thus improving their backscatter enhancement capabilities. Sonication has been found to be a reliable and reproducible technique for preparing standardized echo contrast agent solutions containing uniformly small microbubbles. Bubbles generated with this technique typically range in size from 1 to 15 microns in diameter with a mean bubble diameter of 6 microns (Keller et al. 1986. J. Ultrasound Med. 5:493-498). However, the durability of these bubbles in the blood stream has been found to be limited and research continues into new methods for production of microbubbles. Research has also focused on production of hollow microparticles for use as contrast agents wherein the microparticle can be filled with gas and used in ultrasound imaging. These hollow microparticles, however, also have uses as drug delivery agents when associated with drug products. These hollow microparticles can also be associated with an agent that targets selected cells and/or tissues to produce targeted contrast agents and/or targeted drug delivery agents.

A principal limitation to the clinical utility of microparticles as contrast agents as well as compositions useful in drug delivery is their size. There is a long-felt need in the art for novel, sub-micron diameter echogenic materials that can also be used to deliver a targeted therapeutic agent that can be reliably and easily manufactured. The present invention fills this need.

### SUMMARY OF THE INVENTION

One embodiment of the present invention comprises a method of making polymeric echogenic microcapsules and nanocapsules. The method comprises: (1) emulsifying (e.g., mixing by sonication) an organic phase with a first aqueous phase to provide a first water in oil emulsion; (2) sequentially adding a dose of a second aqueous phase to the first water in oil emulsion until an inversion oil in water emulsion is formed such that from 50 to 99% of a water miscible solvent from the organic phase is extracted from the organic phase into the second aqueous phase; (3) adding water to the oil in water emulsion and thereby further extracting the water miscible solvent and forming polymeric microparticles and nanoparticles; and (4) removing sublimable substances (e.g., by freeze drying) and thereby obtaining polymeric echogenic microcapsules and nanocapsules.

In one aspect, the organic phase comprises a polymer and a non-water soluble sublimable substance which are dissolved in a water-miscible solvent. In another aspect, the first aqueous phase comprises a water soluble sublimable substance dissolved in water. In still another aspect, the second aqueous phase comprises a salting-out agent (or a solvent extracting agent) and a stabilizing agent (colloid) dissolved in water, wherein the stabilizing agent is present in a highly concentrated solution of a salting-out agents or a solvent extracting agents in water. In yet another aspect, the polymer is poly(lactic acid), the non-water soluble sublimable substance is camphor, the water-miscible solvent is acetone, the water soluble sublimable substance is ammonium carbonate, the stabilizing agent is poly(vinyl)alcohol, and the salting out agent is magnesium chloride which is present in at least 50 wt % of the second aqueous phase.

Another embodiment of the present invention comprises a pharmaceutical composition comprising a nanosized contrast agent, wherein the contrast agent is manufactured by a method comprising the steps: (1) emulsifying (e.g., mixing by sonication) an organic phase with a first aqueous phase to provide a first water in oil emulsion; (2) sequentially adding a dose of a second aqueous phase to the first water in oil emulsion until an inversion oil in water emulsion is formed such that from 50 to 99% of a water miscible solvent from the organic phase is extracted from the organic phase into the second aqueous phase; (3) adding water to the oil in water emulsion and thereby further extracting the water miscible solvent and forming polymeric microparticles and nanoparticles; and (4) removing sublimable substances (e.g., by freeze drying) and thereby obtaining polymeric echogenic microcapsules and nanocapsules.

In one aspect, the contrast agent further comprises a targeting moiety. In another aspect, the contrast agent further comprises a therapeutic agent.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of schematic diagrams depicting methods of manufacturing nanocapsules and microcapsules of the present invention. FIG. 1A is a schematic diagram depicting a general salting out method. FIG. 1B is a schematic diagram depicting a modified salting out method for manufacturing the echogenic nanocapsules and microcapsules of the present invention.

FIG. 2, comprising FIG. 2A and FIG. 2B, is a series of images depicting electron micrographs of contrast agents. FIG. 2A depicts microcapsules prepared using 50:50 PLGA-COOH prepared with 0.25M ammonium carbonate encapsulating agent. Magnification 3000×. FIG. 2B depicts PLA-COOH microcapsules prepared with 0.25M Ammonium Carbonate. Magnification 3000×.

FIG. 3 is an electron micrograph depicting PLA-COOH contrast agent prepared with 12 ml methylene chloride. Magnification 1890×.

FIG. 4, comprising FIG. 4A and FIG. 4B, is a series of images depicting electron micrographs of contrast agents. FIG. 4A depicts an AMRAY electron micrograph of a PLA-COOH contrast agent prepared with 15 mil methylene chloride. Magnification 1140×. FIG. 4B depicts an electron micrograph of a PLA-COOH contrast agent, prepared with 17 ml methylene chloride. Magnification 6000×.

FIG. 5 is an AMRAY electron micrograph of a PLA-COOH contrast agent prepared with 20 ml methylene chloride. Magnification 1620×.

FIG. 6, comprising FIG. 6A through FIG. 6D, is a series of images depicting the resulting size distribution of microcapsules and nanocapsules prepared with PLA-COOH prepared with increasing volumes of methylene chloride. FIG. 6A is a graph depicting the size distribution of microcapsules and nanocapsules prepared with 12 ml of methylene chloride. FIG. 6B is a graph depicting the size distribution of microcapsules and nanocapsules prepared with 15 ml of methylene chloride. FIG. 6c is a graph depicting the size distribution of microcapsules and nanocapsules prepared with 17 ml of methylene chloride. FIG. 6D is a graph depicting the size distribution of microcapsules and nanocapsules prepared with 20 ml of methylene chloride.

FIG. 7 is a graph depicting the effects that increasing the organic phase has on the acoustic properties of PLA-COOH microcapsules and nanocapsules prepared with methylene chloride.

FIG. 8, comprising FIG. 8A and FIG. 8B, is a series of graphs depicting the percent dB decay over time for PLA-COOH microcapsules and nanocapsules prepared with varying volumes of methylene chloride. FIG. 8A is a graph depicting the percent dB decay that occurs for nanocapsules and microcapsules prepared with 15 and 12 ml of methylene chloride. FIG. 8B is a graph depicting the percent dB decay that occurs for nanocapsules and microcapsules prepared with 20 and 17 ml of methylene chloride.

FIG. 9 is an image depicting a grey scale ultrasound image of a tumor. A 9.4 mm, B=3.3 mm.

FIG. 10, comprising FIG. 10A and FIG. 10B, is a series of images depicting power Doppler ultrasound images of a tumor. FIG. 10A is an ultrasound image depicting the tumor before the injection of PLA-COOH contrast agent into a rat.

FIG. 10B is an ultrasound image of a tumor after injection of 100 μl of 0.04 g/ml solution of PLA-COOH contrast agent into a rat.

FIG. 11 is a graph depicting the influence of PVA concentration on resulting particle size using the salting out method. Polymer concentration (5.0 wt %), PVA (25 kDa), 2.5 aqueous/organic phase ratio, stirring speed (3500 rpm) held constant.

FIG. 12, comprising FIG. 12A through FIG. 12C, is a series of images depicting the results of dynamic light scattering analysis of PLA nanoparticles. FIG. 12 A is an image depicting the results for PLA nanoparticles prepared with 15 wt % PVA. FIG. 12B is an image depicting the results for PLA nanoparticles prepared with 10 wt % PVA. FIG. 12C is an image depicting the results for PLA nanoparticles prepared with 5 wt % PVA.

FIG. 13, comprising FIG. 13A and FIG. 13B, is a series of graphs depicting the effect increasing the molecular weight of the PVA influences particle size. FIG. 13A depicts Influence PVA molecular weight on the particle size. FIG. 13A is a graph depicting the effect of polymer concentration on particle size. (5.0 wt %), PVA (10 wt %), 2.5 aqueous/organic phase ratio, stirring speed (2000 rpm) held constant. FIG. 13B depicts effect of PLA concentration on the particle size. PVA (10 wt %), aqueous/organic phase ratio (2.5), stirring speed (2000 rpm) held constant.

FIG. 14 is an image depicting a scanning electron micrograph of solid PLA nanocapsules, magnification 2500×. PVA M_{w} (25 kDa) and concentration (10.0 wt %), PLA (5.0 wt %), 2.5 aqueous/organic phase ratio, stirring speed (2000 rpm) held constant.

FIG. 15 an image depicting a scanning electron micrograph of solid PLA nanocapsules. Magnification 8000×. PVA M_{w} (25 kDa) and concentration (10.0 wt %), PLA (5.0 wt %), 2.5 aqueous/organic phase ratio, stirring speed (200 rpm) held constant.

FIG. 16 is an image depicting the results of dynamic light scattering analysis of PLA nanoparticles prepared with PVA (25 kDa, 10 wt %), PLA (5 wt %), 2.5 aqueous/organic ratio, and 2000 rpm stirring speed. Intensity v. diameter is shown.

FIG. 17, comprising FIG. 17A through FIG. 17D, is a series of graphs depicting particle size analysis of PLA nanocapsules manufactured with varying PLA concentrations. Polymer concentration (5.0 wt %), PVA (25 kDa), 0.04 g camphor, 1M Ammonium Carbonate, 2.5 aqueous/organic phase ratio, stirring speed (2000 rpm) held constant. Graphs display number vs. size. FIG. 17A is a graph depicting the size distribution of nanocapsules produced using 15 wt % PVA. FIG. 17B is a graph depicting the size distribution of nanocapsules produced using 10 wt % PVA. FIG. 17C is a graph depicting the size distribution of nanocapsules produced using 5 wt % PVA. FIG. 17D is a graph depicting the size distribution of nanocapsules produced using 2 wt % PVA.

FIG. 18 is a graph depicting the dose response curve of PLA nanocapsules prepared with varying PVA concentrations. PLA (5 wt %), PVA (25 kDa), 0.04 g camphor, 1.0M ammonium carbonate, 2.5 aqueous/organic, 2000 rpm stirring. Dose-response curves generated at 37° C. with a 5 MHz transducer.

FIG. 19 is a graph depicting the relationship between dB enhancement as a function of mean particle size of the contrast agent prepared using the salting out procedure. PLA (5 wt %), PVA (25 kDa), 0.04 g camphor, 1.0M ammonium carbonate, 2.5 aqueous/organic, 2000 rpm stirring. Dose-response curves generated at 37° C. with a 5 MHz transducer.

FIG. 20 is a graph depicting the time decay of PLA nanocapsules manufactured with varying amounts of PVA concentrations. PLA (5 wt %), PVA (25 kDa), 0.04 g camphor, 1.0M ammonium carbonate, 2.5 aqueous/organic, 2000 rpm stirring. Dose-response curves generated at 37° C. with a 5 MHz transducer.

FIG. 21, comprising FIG. 21A through FIG. 21D, is a series of graphs depicting the dose response curves of nanocapsules manufactured with varying concentrations of PLA. FIG. 21A is a graph depicting the dose response curve for PLA (2, 5, and 10 wt %), PVA (15 wt %) 0.04 g camphor, 1.0M ammonium carbonate, 2.5 aqueous/organic, 2000 rpm stirring. Dose-response curves generated at 37° C. with a 5 MHz transducer. FIG. 21B is a graph depicting the dose response curve for PLA (2, 5, and 10 wt %), PVA (10 wt %) 0.04 g camphor, 1.0M ammonium carbonate, 2.5 aqueous/organic, 2000 rpm stirring. Dose-response curves generated at 37° C. with a 5 MHz transducer. FIG. 21C is a graph depicting the dose response curve for PLA (2, 5, and 10 wt %), PVA (5.0 wt %) 0.04 g camphor, 1.0M ammonium carbonate, 2.5 aqueous/organic, 2000 rpm stirring. Dose-response curves generated at 37° C. with a 5 MHz transducer. FIG. 21D is a graph depicting the dose response curve for PLA (2, 5, and 10 wt %), PVA (2 wt %) 0.04 g camphor, 1.0M ammonium carbonate, 2.5 aqueous/organic, 2000 rpm stirring. Dose-response curves generated at 37° C. with a 5 MHz transducer.

FIG. 22 is a graph depicting the time decay of PLA nanocapsule with varying concentrations of PLA. PVA (5 wt %) 0.04 g camphor, 1.0M ammonium carbonate, 2.5 aqueous/organic, 2000 rpm stirring. Dose-response curves generated at 37° C. with a 5 MHz transducer.

FIG. 23 is an image depicting a scanning electron micrograph of PLA contrast agents prepared using the salting our procedure. Magnification=6000×. Size bar=5 μm. PVA (MW=25 kDa) and 5.0 wt % concentration, PLA (5 wt %), 2.5 aqueous/organic phase ratio, stirring speed 2000 rpm, held constant.

### DETAILED DESCRIPTION OF THE INVENTION

The present invention is based on the discovery of a novel method of producing a nanosized echogenic contrast agent (CA) useful in both ultrasonic imaging and drug delivery. The method of the invention comprises a salting out step to produce a polymeric nanosized CA. Accordingly, the present invention encompasses a novel, nanosized polymeric CA, methods of producing a polymeric nanosized CA, and methods of using a polymeric nanosized CA.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The “salting out” method is a method of separation. The “salting out” component of the method of the invention involves highly saturating the aqueous phase II with salt causing the water molecules to be more attracted by the salt ions in comparison to the acetone. The acetone therefore becomes less soluble in water, producing a two phase system. Advantageously, porogens introduced to the organic phase and to a first aqueous phase did not interfere with this process and provided a synergistic result where echogenic nanoparticles were obtained.

The term “a salting-out agent” or “a solvent extracting agent” as used herein denotes chemical compound, e.g., a salt, capable of causing separation (i.e., extraction) of a water miscible solvent (e.g., acetone) from the organic phase when introduced as a part of an aqueous phase to a water/oil emulsion containing the organic phase. Exemplary salting-out agents or solvent extracting agents include CaCl₂, MgCl₁₂*6H₂O, sucrose, Mg acetate and those salts listed in U.S. Pat. No. 4,968,350. The choice of salting-out agents or solvent extracting agents is important as can be seen from the following four class characterization by (Ibrahim, 1989) according to the behavior of their saturated or highly concentrated aqueous solution when brought in contact with acetone:

Class A: no liquid-liquid phase separation but precipitation of the solute in the organic-aqueous medium (e.g., calcium sulfate, magnesium sulfate, ammonium nitrate, potassium sulfate, potassium chloride, potassium nitrate, sodium sulfate and sodium nitrate);

Class B: no liquid-liquid phase separation, regardless of the acetone-water volume ratio (e.g., aluminum nitrate, calcium nitrate and magnesium nitrate), The solutes were soluble in the organic-aqueous phase but did not produce the salting-out of acetone from water.;

Class C: liquid-liquid phase separation accompanied by a partial precipitation of the salting-out agent in the medium (e.g., aluminum sulfate, ammonium sulfate, ammonium chloride and sodium chloride). An upper acetone phase and a precipitate were observed. For some solutes like sodium chloride, it should be noted that lower concentrations effected phase separation without precipitation. Thus, the classification of some compounds of class C may be changed to class D, when lower salt concentrations are considered. Aluminum sulfate and ammonium sulfate produced liquid-liquid phase separation but the initial volume of the upper phase was larger than the acetone volume introduced into the mixture and furthermore, this volume decreased by increasing salt concentration; and

Class D: liquid-liquid phase separation without formation of a precipitate (e.g., aluminum chloride, calcium chloride and magnesium magnesium chloride). Five compounds, all from class C and D, showed potential properties for producing the salting-out of acetone from water. They were all chloride derivatives.

Examples of polymers that can be used in this method include, but are not limited to, poly(lactic acid), poly(lactide), a poly(glycolide), a poly(caprolactone), a copolymer of poly(lactide) and poly(glycolide), a copolymer of lactide and lactone, a polysaccharide, a poly(anhydride), a poly(styrene), a poly(alkylcyanoacrylate), a poly(amide), a poly(phosphazene), a poly(methylmethacrylate), a poly(urethane), a copolymer of methacrylic acid and acrylic acid, a copolymer of hydroxyethylmethacrylate and methylmethacrylate, a poly(aminoacid), and a polypeptide. Preferred polymers are those which are biocompatible and/or biodegradable. In a preferred embodiments the polymer is poly(D,L-lactic acid) or poly(D,L-lactide).

Examples of water-miscible solvents include acetone, tetrahydrofuran, acetonitrile, ethyl acetate, and isopropanol and those solvents listed in U.S. Pat. No. 4,968,350.

The term “porogen” as used herein denotes a sublimable substance (water soluble and/or non-water soluble) which leaves pores after it is removed (sublimed, freeze dried, etc.).

The term “non-water soluble substance” as used herein denotes a substance which dissolves in a non-polar solvent and is capable of subliming from a solid state. Examples include but are not limited to, camphor, camphene, naphthalene, cocoa butter and theobroma oil.

The term “water soluble sublimable substance” as used herein denotes a substance which dissolves in a polar solvent and is capable of subliming from a solid state. Examples include but are not limited to, ammonium carbonate and other ammonium salts, theobromine and theobromine acetate.

“Stabilizing agents” are those which act as a protective hydrocolloid, at both the product preparation stage and the finished product stage, once the finished product has been re-dispersed in an aqueous medium. A water-soluble macromolecular polysaccharide such as gum arabic or gum tragacanth or a water-soluble polypeptide such as gelatin can be used as this substance. In a preferred example, a water-soluble polymer of synthetic origin, e.g., poly(vinyl) alcohol, is used.

As used herein, a “targeting moiety” refers to a molecule that binds specifically to a molecule present on the cell surface of a target cell.

By the term “specifically binds,” as used herein, is meant a molecule, such as an antibody, which recognizes and binds to a cell surface molecule or feature, but does not substantially recognize or bind other molecules or features in a sample.

### Description:

### I. Compositions

In one embodiment, the present invention encompasses an echogenic polymeric microcapsule and nanocapsule produced according to the methods of the present invention. For purposes of the present invention, by “nanocapsule” it is meant a capsule sufficiently small in size to access the microvasculature of the human body. Nanocapsules of the present invention range in size from about 10 nm to about 500 nm, while microcapsules of the present invention range in size from about 500 nm to about 1000 microns. Nanocapsules of this size provide an advantage in that they can access areas difficult if not impossible to reach with microcapsules. For example, nanocapsules can pass through leaky tumor vasculature. In addition, nanocapsules have different resonance frequencies thus providing advantages in both imaging and delivery of bioactive agents. Nanocapsules of the present invention have been found to be echogenic above 10 MHZ.

Thus, as demonstrated herein, in another embodiment, echogenic polymer microcapsules and nanocapsules produced in accordance with these methods can be used for imaging of any of the various tissues and epithelium and/or endothelium thereof routinely imaged with ultrasound techniques including, but not limited to, renal tissue, brain tissue, tumor vasculature, skin tissue, pancreatic tissue, breast tissue, heart tissue, prostate tissue, uterine tissue, adrenal gland tissue, retinal tissue, muscle tissue, areas of plaque and areas of ischemia.

For use as a contrast agent, it is preferred that the echogenic microcapsules and/or nanocapsules of the present invention be hollow or porous so that they can be filled with gas. Such gas-filled polymer microcapsules are produced by bringing echogenic hollow or porous polymer microcapsules into contact with a gas and equilibrating the microcapsules with the gas for a period of time sufficient to allow diffusion of the gas into the polymer microcapsules, resulting in a gas-filled polymer microcapsule. This procedure of exposing hollow or porous polymer microcapsules to the gas may be carried out at ambient pressure (atmospheric), at subatmospheric pressure, or at an elevated pressure. The period of time required to effect filling of the hollow microcapsules with the gas is relatively short, typically requiring only a few minutes, the actual time depending on the manner and pressure at which the hollow microcapsules are equilibrated with the gas.

The term “gas” as used in this specification includes substances which are in gaseous form under normal storage conditions, e.g., at about 15 to 25° C., and/or at normal mammalian body temperature, e.g., 37° C. in humans. The resulting gas-filled polymer microcapsules of this invention may be stored as a dry, free-flowing powder, preferably in the presence of the gas contained in the polymer microcapsules.

The gas-filled microcapsules and nanocapsules are useful as contrast agents in medical imaging, such as diagnostic ultrasound. Ultrasound contrast compositions typically comprise the hollow or porous polymer microcapsules or nanocapsules, filled with a gas, and dispersed in an aqueous liquid which serves as a carrier for the contrast agent. Aqueous liquids that can be used include, but are not limited to, isotonic saline and phosphate-buffered saline. The contrast agent composition is then injected into the bloodstream and used for ultrasound visualization of specific blood vessels or body organs.

In yet another embodiment, the polymeric echogenic microcapsules and nanocapsules of the present invention can be used for delivery of bioactive agents. In this embodiment, a bioactive agent may be adsorbed to and/or attached to the surface of the microcapsule or nanocapsule. To adsorb a drug or therapeutic agent to the microcapsule/nanocapsule surfaces, the drug is dissolved in distilled water or a buffer, and then the dried microcapsules/nanocapsules are suspended in distilled water with the drug. The suspension is stirred overnight and then the suspension centrifuged to collect capsules.

The resulting microcapsules/nanocapsules are then washed, frozen and lyophilized. The lyophilized microcapsules/nanocapsules have the drug product to be delivered adsorbed to their surfaces. Bioactive agents can also be attached to the microcapsules and/or nanocapsules in accordance with well known methods for conjugation. For example, a conjugation method may be used substituting the bioactive agent for the RGD peptide. Alternatively, or in addition, a bioactive agent can be encapsulated in the microcapsule or nanocapsule. Water soluble bioactive agents can be encapsulated in the microcapsules or nanocapsules by including water during emulsification and dissolving the bioactive agent in this water. Non-water soluble bioactive agents can be encapsulated in the microcapsules or nanocapsules by dissolving the bioactive compound in the non-polar organic solvent in the first step of preparation of these capsules.

Examples of bioactive agents which can be adsorbed, attached and/or encapsulated in the microcapsules and/or nanocapsules of the present invention include, but are not limited to, antineoplastic and anticancer agents such as azacitidine, cytarabine, fluorouracil, mercaptopurine, methotrexate, thioguanine, bleomycin peptide antibiotics, podophyllin alkaloids such as etoposide, VP-16, teniposide, and VM-26, plant alkaloids such as vincristine, vinblastin and paclitaxel, alkylating agents such as busulfan, cyclophosphamide, mechlorethamine, melphanlan, and thiotepa, antibiotics such as dactinomycin, daunorubicin, plicamycin and mitomycin, cisplatin and nitrosoureases such as BCNU, CCNU and methyl-CCNU, anti-VEGF molecules, gene therapy vectors and peptide inhibitors such as MMP-2 and MMP-9, which when localized to tumors prevent tumor growth.

Once prepared, microcapsules and/or nanocapsules comprising the bioactive agent can be suspended in a pharmaceutically acceptable vehicle for injection into animals, including humans. Once injected, the bioactive agent is released by either biodegradation over time of the polymer microcapsule structure, by initiation of release of the bioactive agent through exposure to ultrasound, or by a combination thereof.

In another embodiment, the microcapsules and/or nanocapsules of the present invention can be used to direct delivery of a bioactive agent to any of the various tissues and epithelium and/or endothelium thereof including, but not limited to, renal tissue, lung tissue, brain tissue, tumor vasculature, skin tissue, pancreatic tissue, breast tissue, heart tissue, prostate tissue, intestinal tissue, uterine tissue, adrenal gland tissue, retinal tissue, muscle tissue, areas of plaque, areas of inflammation, and areas of ischemia.

The microcapsules and/or nanocapsules of the present invention may further comprise a targeting moiety attached to the capsule surface which upon systemic administration can target the contrast agent or the delivery agent to a selected tissue or tissues, or cell in the body.

### Targeting Moieties

A targeting moiety may be an antibody, a naturally-occurring ligand for a receptor or functional derivatives thereof, a vitamin, a small molecule mimetic of a naturally-occurring ligand, a peptidomimetic, a polypeptide or aptamer, or any other molecule provided it binds specifically to a cell surface molecule, or a fragment thereof. Any cell surface molecule may be targeted provided binding of the targeted CA is specific. Cell surface molecules that may be targeted include, but are not limited to, cell adhesion molecules (CAM), glycosylphosphatidylinisotol (GPI)-anchored proteins, receptors, including but not limited to hormone receptors (e.g., epidermal growth factor receptor), sugar receptors (e.g., mannose receptor and lectin receptor), glutamate receptor mGluR5, gamma c cytokine receptor, TGF-β receptor, neurotransmitter and neuropeptide receptors, ion channels, comprising voltage- and ligand-gated ion channel.

Further examples of targeting moieties useful in the present invention include, but are in no way limited to, RGD which binds to integrins on tumor blood vessels, NGR motifs which bind to aminopeptidase N on tumor blood vessels and ScFvc which binds to the EBD domain of fibronectin. Accordingly, targeting moieties can be routinely selected so that a contrast agent or delivery agent of the present invention, or a combination thereof, is directed to a desired location in the body such as selected tissue or tissues, cells or an organ, or so that the contrast agent or delivery agent of the present invention can distinguish between various tissues such as diseased tissue versus normal tissue or malignant tissue versus benign tissue. Targeted contrast and/or delivery agents can be administered alone or with populations of contrast agents and/or delivery agents of the present invention which do not further comprise a targeting moiety.

A targeting moiety may be coupled to a CA of the invention by any means known in the art. By way of a non-limiting example, coupling involves forming ester, thioester, amide, or sulfamide linkages. Coupling hydroxy, thio, or amine groups with carboxy or sulfoxy groups is known to those skilled in the art.

The polymers can contain various functional groups, such as hydroxy, thio, and amine groups, that can react with a carboxylic acid or carboxylic acid derivative under the coupling conditions. Reactive functional groups not involved in the coupling chemistry must be protected to avoid unwanted side reactions. After the carboxylic acid or derivative reacts with a hydroxy, thio, or amine group to form an ester, thioester, or amide group, any protected functional groups can be deprotected by means known to those skilled in the art.

The term “protecting group” as used herein refers to a moiety which blocks a functional group from reaction, and which is cleavable when there is no longer a need to protect the functional group. Suitable protecting groups for the hydroxyl group include, but are not limited to, certain ethers, esters and carbonates (Greene, T. W. and Wuts, P. G. M., “Protective groups in organic synthesis,” John Wiley, New York, 2nd Ed. (1991)). Suitable protecting groups for the carboxyl group include, but are not limited to, those described in Green and Wuts, Protecting Groups in Organic Synthesis, John Wiley (1991). Side-chain functionalities such as carboxylic acids, alcohols, and amines may interfere with the coupling chemistry and must be appropriately protected.

As used herein, “side-chain functionality” refers to functional groups, such as hydroxy, thio, amine, keto, carboxy, alkenyl, alkynyl, carbonyl, and phosphorus derivatives such as phosphate, phosphonate and phosphinate in the polymer or material to be covalently attached to the polymer, that is not involved in coupling to form an ester, thioester, amide or sulfamide bond. Examples of suitable protecting groups are well known to those skilled in the art. See, generally, Greene and Wuts, Protecting Groups in Organic Chemistry, John Wiley (1991). Examples of protecting groups for amine groups include, but are not limited to, t-butyloxycarbonyl (Boc), benzyloxycarbonyl (Cbz), o-nitrobenzyloxycarbonyl, and trifluoroacetamide (TFA).

### Targeting Moiety—Antibodies

When the antibody used as a targeting moiety in the compositions and methods of the invention is a polyclonal antibody (IgG), the antibody is generated by inoculating a suitable animal with the targeted cell surface molecule. Antibodies produced in the inoculated animal which specifically bind to the cell surface molecule are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1998, In: Using Antibodies, A Laboratory Manual, Cold Spring Harbor, N.Y.).

Monoclonal antibodies directed against a full length targeted cell surface molecule or fragments thereof may be prepared using any well known monoclonal antibody preparation procedures, such as those described, for example, in Harlow et al. (1998, In: Using Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a targeted cell surface molecule, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with targeted cell surface molecule epitopes. These antibodies are capable of binding to the targeted cell surface molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the targeted cell surface molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1988, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as targeting moieties in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Samples may need to be modified in order to render the target molecule antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

One of skill in the art will appreciate that it may be desirable to detect more than one protein of interest in a biological sample. Therefore, in particular embodiments, at least two antibodies directed to two distinct proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

### Targeting Moieties-Protein, Peptide, and Polypeptide

Targeting moieties useful in the invention may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a targeting moiety, a peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and other peptide targeting moieties may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### II. Methods

In creating a contrast agent, there must be an impedance mismatch between the suspending fluid and the agent to reflect the ultrasound signal. To accomplish this, a sublimable substance (i.e., a porogen, water soluble and/or non-water soluble) is added to aqueous and the organic phases and later removed through lyophilization, leaving a void.

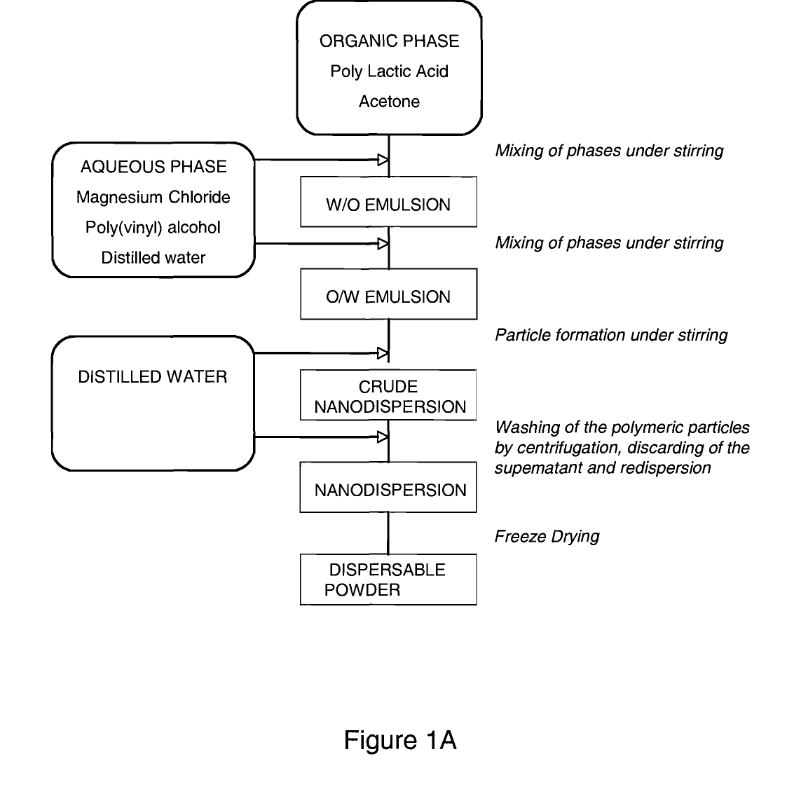

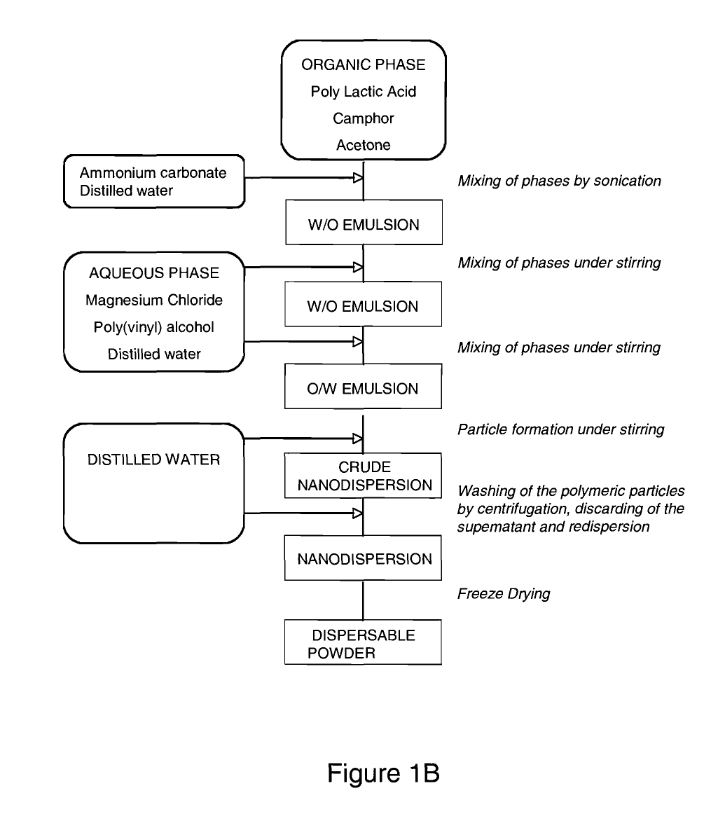

In the method of the invention, the salting out procedure as shown in FIG. 1A was modified to produce echogenic capsules.

The “salting out” method is a method of separation. The “salting out” component of the method of the invention involves highly saturating the aqueous phase II with salt causing the water molecules to be more attracted by the salt ions in comparison to the acetone. The acetone therefore becomes less soluble in water, producing a two phase system. Advantageously, porogens introduced to the organic phase and to a first aqueous phase did not interfere with this process and provided a synergistic result where echogenic nanoparticles were obtained.

Examples of polymers that can be used in this method include, but are not limited to, poly(lactic acid), poly(lactide), a poly(glycolide), a poly(caprolactone), a copolymer of poly(lactide) and poly(glycolide), a copolymer of lactide and lactone, a polysaccharide, a poly(anhydride), a poly(styrene), a poly(alkylcyanoacrylate), a poly(amide), a poly(phosphazene), a poly(methylmethacrylate), a poly(urethane), a copolymer of methacrylic acid and acrylic acid, a copolymer of hydroxyethylmethacrylate and methylmethacrylate, a poly(aminoacid), and a polypeptide. Preferred polymers are those which are biocompatible and/or biodegradable. In a preferred embodiments the polymer is poly(D,L-lactic acid) or poly(D,L-lactide).

Examples of water-miscible solvents include acetone, tetrahydrofuran, acetonitrile, ethyl acetate, and isopropanol and those solvents listed in U.S. Pat. No. 4,968,350.

The method of the invention comprises the following steps: (1) emulsifying (e.g., mixing by sonication) an organic phase with a first aqueous phase to provide a first water in oil emulsion, (2) sequentially adding a dose of a second aqueous phase to the first water in oil emulsion until an inversion oil in water emulsion is formed such that from 50 to 99% of a water miscible solvent from the organic phase is extracted from the organic phase into the second aqueous phase, (3) adding water to the oil in water emulsion and thereby further extracting the water miscible solvent and forming polymeric nanoparticles, (4) removing sublimable substances (e.g., by freeze drying) and thereby obtaining echogenic polymeric nanoparticles.

An organic phase comprises a polymer and a non-water soluble sublimable substance which are dissolved in a water-miscible solvent. Various ratios of aqueous/organic, PVA concentrations and PLA concentrations can be used depending on application.

In one embodiment, the organic phase comprises camphor (10% w/w of polymer) and PLA 100DL (end-capped) dissolved in acetone.

A first aqueous phase comprises a water soluble sublimable substance dissolved in water.

A second aqueous phase comprises a salting-out agent (or a solvent extracting agent) and a stabilizing agent (colloid) dissolved in water. The stabilizing agent (e.g., poly(vinyl alcohol) (PVA) is present in a highly concentrated solution (e.g., from 50-100% of the concentration needed to achieve a saturated solution] of a salting-out agent or a solvent extracting agent in water.

Prior to the emulsification of the organic phase and the second aqueous phase, 1 ml of a 1.0M ammonium carbonate solution (the first aqueous phase) is first emulsified for example by sonication at 110 Watts for 30 seconds, in the organic phase.

In another embodiment, the second aqueous phase was prepared as follows:

(i) 60.0 wt % magnesium chloride hexahydrate (MgCl₂*6H₂O)

(ii) 5.0 wt % PVA, and

(iii) 35.0 wt % distilled deionized water,

wherein salt concentration was held constant at 60 wt % of aqueous phase.

The second aqueous phase (20 g) is then added drop-wise to the first emulsion under a mechanical stirrer (Caframo BD6015 bench top) at 200 rpm with a 3-blade propeller for ˜10 minutes. A sufficient amount of water (˜50 ml) is then added under stirring to cause acetone diffusion and creation of nascent nanoparticle suspension. The second aqueous phase consists of 60% w/w magnesium chloride hexahydrate, 5.0 wt % PVA, and de-ionized water. Aqueous to organic weight ratio is held constant at 2.5.

The nanocapsules are collected and washed by centrifugation for 20 minutes (3×) at 15,000 rpm (˜30,000 g) to remove the salt, excess organic solvent and PVA. Particles are then resuspended in deionized water, frozen at −80° C., and lyophilized for 48 hours to remove camphor and ammonium carbonate.

It is desirable to prepare the second aqueous phase ahead of time due to the heat dependency to dissolve PVA. The following procedure can be adapted in preparation of the second aqueous phase: combine water and MgCl₂*6H₂O in a beaker containing a magnetic stir bar and stir on a hot/stir plate; dissolve the salt in the water while increasing the temperature of the solution to about 80° C.; weigh out the desired amount of PVA and once the salt solution reaches the correct temperature, slowly add the PVA while stirring to prevent clumping; stir for about 3 hours at constant temperature and add water to adjust for evaporation; after 3 hours, cool and add water or reheat if needed depending on the amount of evaporation of water.

The preferred embodiment will now be described in detail (see FIG. 1B)

1) Prepare the organic phase as follows:

(a) 2.0 wt % polymer, (b) acetone, and (c) non-water soluble sublimable substance, e.g., camphor (10 wt % of polymer).

2) Combine the camphor, polymer, and acetone in a beaker (50 ml max volume) with a stir bar and stir on a magnetic stir plate, covered with parafilm to prevent evaporation, until camphor and polymer are dissolved.

3) While the PLA and camphor are dissolving in acetone, weigh out 20 g of the aqueous solution initially prepared. Load two 10 ml syringes with equal amounts of aqueous solution and place aside until step 5. Additionally, prepare a 1.0M solution of ammonium carbonate.

4) When the camphor and polymer are fully dissolved, remove stir bar and add 1 ml of 1.0 M ammonium carbonate solution and sonicate at 110 W for 30 seconds pulsing 3 seconds on and 1 second off.

5) After sonication, pour the contents into a 250 ml beaker which should already be placed under stirrer. Under mechanical stirring (2000 rpm), take the two syringes and add the aqueous to the organic phase dropwise. Let emulsion stir for about 8-10 minutes.

6) After the time elapsed, add sufficient amount of deionized water (˜50 ml) quickly (e.g., 5 sec) to allow the diffusion of acetone into the aqueous phase resulting in the formation of nanoparticles.

Add at least an equal amount of water that matches the weight prepared of the aqueous phase.

7) Combine and purify nanocapsules by ultracentrifugation for 20 min at 15,000 rpm (30,000 g). Discard the supernatant and resuspend the nanocapsules in distilled water. Repeat this process of centrifugation 3 times to thoroughly remove the organic solvent, free PVA, and electrolytes.

8) After combining and purifying capsules, resuspend nanocapsules in distilled water, place Kim wipe on top of tube with rubber band, freeze at −80° C. for at least 30 min or until thoroughly frozen.

9) Put the frozen nanocapsules in a freeze dryer vessel and on the freeze dryer for at least 48 hours.

10) Store nanocapsules powder at −20° C. in sealed container.

A porogen substance can be added to either or both aqueous and organic phases. In a preferred embodiment, the water soluble sublimable substance such as, for example, ammonium carbonate, is added to the first aqueous phase and the non-water soluble sublimable substance such as camphor is added to the organic phase.

The advantage of using the salting out method provides an enhanced control over the final particle characteristics. It presents the ability to use other miscible solvents, salting out agents, and polymers allowing the process to be further controlled and tune to a specific application of the capsule. For example, when the ultimate goal is to incorporate a bioactive molecule or therapeutic agent, the process parameters can be altered to promote drug loading. A person skilled in the art would appreciate that the method of the invention can be further optimized by, for example, selecting other organic solvents, salting out agents, polymers such as PLGA, variable MW of PLA, and stirring blade geometry.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Polymers

Poly (D,L-lactic acid) without lauryl ester end group (acid end group), (3.5 A lot D02006) was purchased from Absorbable Polymers International, Pelham, Ala. Poly (D,L-lactide) 100 DL Low IV (Lakeshore Biomaterials, lot W2297-587) were purchased from Alkermes, Cincinnati, Ohio. The molecular weights and glass transition temperatures of these polymers are recorded in Table X.

TABLE 1
Polymer molecular eight and glass transition temperature

| Polymer formulation | Molecular | Glass transition |
|---|---|---|
| (lactic acid:glycolic acid) | weight (kDa) | temperature (° C.) |
| 100:0 | 83.0 | 49.16 |
| 100:0 (—COOH) | 36.8 | 50-60 |
| 50:50 | 51.0 | 45.0 |

### Sublimable Agents

Pharmaceutical grade ammonium carbonate and ammonium carbamate were purchased from J. T. Baker, Phillipsburg, N.J. while (1R)-(+)-camphor was purchased from Sigma Chemical Co., St.Louis, Mo.

### Other Chemicals

Poly (vinyl alcohol) (PVA), 88% mole hydrolyzed with a molecular weight range of 13000-23000 Da, 6000 Da, 25,000 Da was purchased from Polysciences Inc, Warrington Pa. PVA (Mowiol 4-88 and 8-88) also having 88% mole hydrolyzed with a molecular weight of 31,000 Da and 67,000 Da respectively and magnesium chloride hexahydrate were purchased from Sigma Aldrich (Fluka) (St. Louis, Mo.). Acetone, hexane, and methylene chloride were purchased from Fisher Scientific, Springfield, N.J.

### Methods of Characterization of Microcapsules and Nanocapsules

### 1. In Vitro Dose and Time Response

To determine the ability of the nanocapsules and/or microcapsules to generate the backscatter of the ultrasound, in vitro tests were conducted in a 100 ml custom-made vessel equipped with an acoustic window, containing 50 ml of phosphate buffered saline (PBS). The freeze dried microcapsules and/or nanocapsules were weighed and suspended in PBS. The suspension was placed in an acoustic testing apparatus which consisted of a 50.8 mm spherically focused transducer (0.75 in diameter) obtained from Panametrics (Waltham, Mass.). 5 MHz frequency was chosen based upon previous studies (El Sheriff and Wheatley, 2003, J. Biomed. Matter Res. 66:347-355).

The transducer was placed in the deionized water bath and focused through the acoustic window of the sample vessel. A pulsar/receiver were used to pulse the transducers at a pulse repetition frequency of 100 Hz. The received signals were amplified and acoustic enhancement was analyzed using a LabView program. Cumulative dose response curves of microcapsules (0.0 mg/ml to 0.36 mg/ml) or nanocapsules (0.0 mg/ml to 0.5 mg/ml) were constructed at an in vitro PBS temperature of 37° C. and pH 7.4.

To determine the stability of the CA under continuous insonation over time, decay in acoustic enhancement was observed using the lowest steadt state dose from the dose response curve. Once a minute for fifteen minutes, readings were taken and analyzed. Results were normalized with time zero have a value of one (1), to represent decay.

### Environmental Scanning Electron Microscopy

A Phillips XL-30 scanning electron microscope was used to image surface morphology of microcapsules and nanocapsules. Freeze dried samples were mounted on a metal stub using double sided tape and sputter coated prior to viewing.

### AMRAY Scanning Electron Microscope

Samples were images using an AMRAY 1830 Scanning Electron Microscope. Freeze dried samples were mounted on a metal stub using double sided tape. Platinum was used to sputter coat microcapsules and nanocapsules prior to viewing.

### Gel Permeation Chromatography

To investigate the effects of process parameters on the fabrication of microcapsules and nanocapsules, gel permeation chromatography (GPC) was performed. GPC can be employed to analyze the molecular weight distribution of an organic soluble polymer. The molecule weight of PLA was determined before and after the double emulsion method.

### Dynamic Light Scattering

A Brookhaven Instruments Dynamic Light Scattering 90 Plus Particel Size Analyzer was used to determine the relative size of the capsules. A concentration of 1 μg/ml was prepared of capsules in PBS filtered through a 0.2 μm filter. The suspension was placed on a vortex mixer to shake for about 2 minutes or until fully dispersed. The samples were run at 25° C., 678 nm wavelength (90 degrees). For each sample, the analysis was done a minimum of three times until the sample stabilized. The average maximum peak is reported.

### Zeta Sizer

A Malvern Zeta Sizer (nano series) particle size analyzer was used to determine size and zeta potential of capsules. A concentration of 1 μg/ml was prepared of capsules in PBS filtered through a 0.2 μm filter. The suspension was placed on a vortex mixer to shake for about 2 minutes or until fully dispersed. The samples were run at 25° C., using a polystyrene latex refractive index of 1.590. The Z average is reported.

The results of the experiments presented in this Example are now described.

In solving the problem of obtaining nanosized CA by the method of the present invention, the following problems had to be solved: the state of the art on parameters to produce solid nanoparticles in the desired size range was inconsistent; adding porogens changed the size of the capsules; adding progen did not always give highly echogenic capsules, the other parameters such as PVA concentration had to be reassessed; and adding porogen increased the polydispersity of the resulting capsule.

The concentration of the protective surfactant colloid (e.g., poly(vinyl alcohol)) and the concentration of polymer also affected size, stability, longevity, and echogenicity of the capsule.

In certain embodiments of the method of the invention, solid capsules were first made using poly (DL-lactic acid) with an ester end cap as a polymer. Due to conflicting data in the art in discussing the influences of process parameters, the salting out method used to produce particles was investigated to determine the specific influences of experimental parameters in achieving the desired size. Numerous factors such as aqueous/organic phase ratio, PVA concentration and molecular weight, PLA concentration, and stirring speed were individually varied to establish the methods of the present invention and thereby obtain a nanosized polymeric CA.

### Example 1

### PLA-COOH Microcapsules

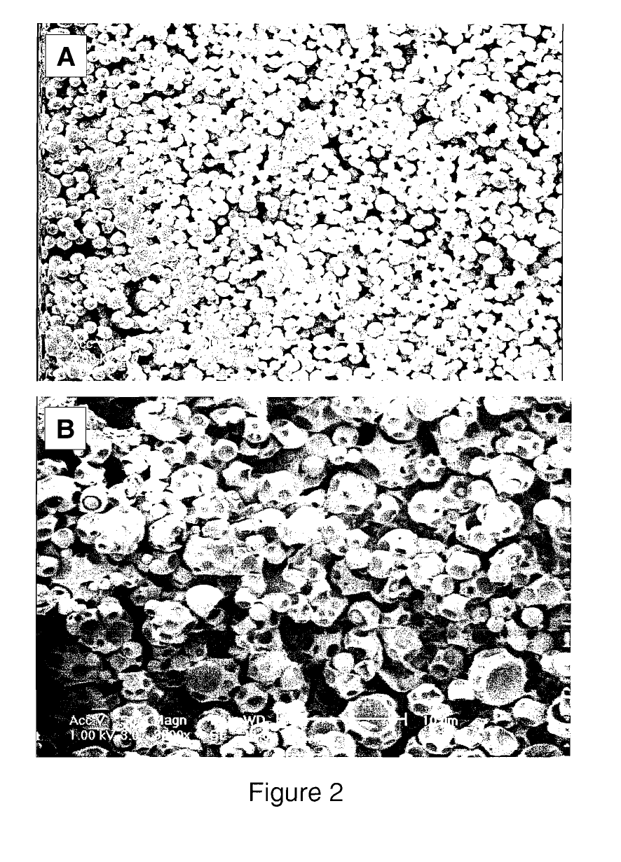

CAs made using 50:50 PLGA-COOH resulted in well rounded capsules of around 1.21 μm diameter with smooth surfaces and highly echogenic (greater than 20 dB enhancement at a dose of 0.003 mg/ml). The current double emulsion method was then used to produce microcapsules composed of PLGA 75:25, 85:15 and 100:0 which were all successful but all had a laryl ester end cap. Since an acid end group has the potential of offering a better substrate for ligand attachment than the end-capped polymer, the investigation of fabrication of CA using PLA-COOH was undertaken. Previous experiments concluded that as the concentration (1.0M, 0.75M, 0.5M, to 0.25M) of the sublimable core (ammonium carbonate) decreased, the resulting capsules exhibited increasingly improved spherical morphology (using 50:50 PLGA-COOH for comparison) (FIG. 2A and FIG. 2B).

The capsules appeared to be larger and very indented in comparison to the 50:50 PLGA-COOH, which is suitable for a drug delivery vehicle, but decreases efficiency for targeting because the ligand will rest inside the pores and not on the surface of the microcapsule. It was hypothesized that the ammonium carbonate might be interacting with the free carboxylic acid group of the polymer and hence the decrease in concentration of ammonium carbonate. Therefore, taking into account the previous results directed to obtain the desired smooth surface morphology, an ammonium concentration of 0.25M was held constant, and other parameters were varied to produce PLA-COOH microcapsules.

a. Effect of Increasing Outer Organic Phase on Porosity

The synthesis of PLA-COOH microcapsules with a smooth surface is of interest. It has been reported that the volume in the organic phase can have an influence on the capsule morphology (Cui et al, 2005, J. Biomed. Mater. Res. B. Appl. Biomater. 73:171-178). The protocol used employs a volume of 10 ml of methylene chloride. Various increasing amounts of methylene chloride (12 ml, 15 ml, 17 ml, and 20 ml) were used in the preparation of PLA-COOH microcapsules to determine the effect on the morphology of the capsule. A concentration of 0.25 M ammonium carbonate was held constant.

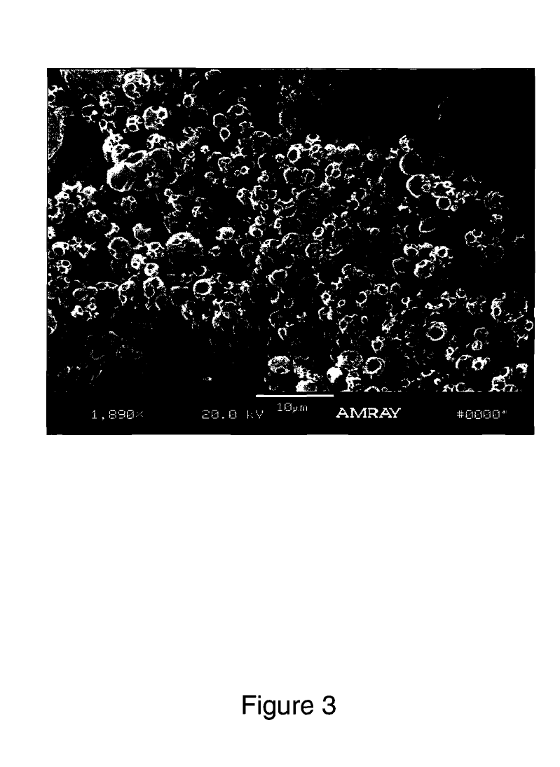

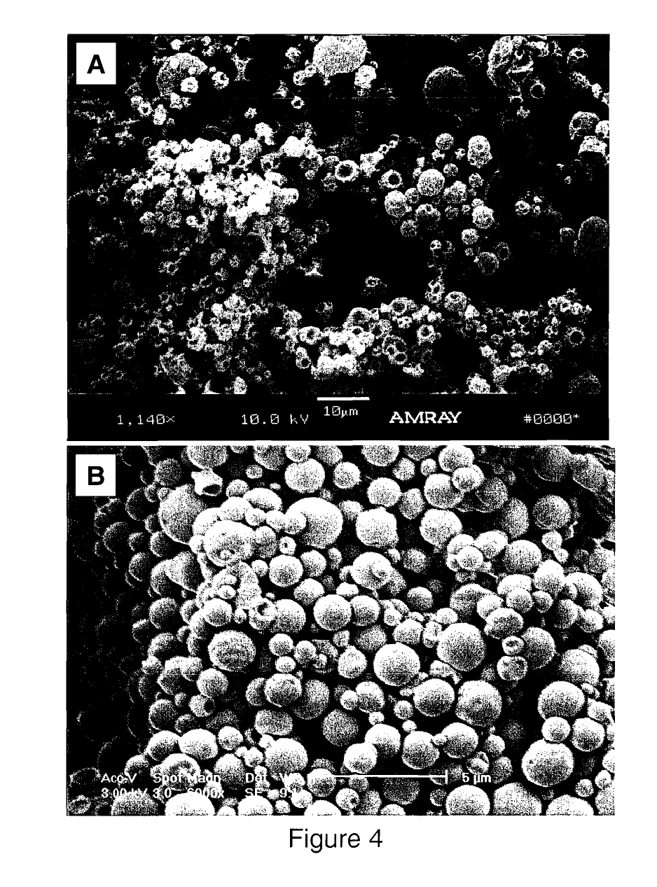

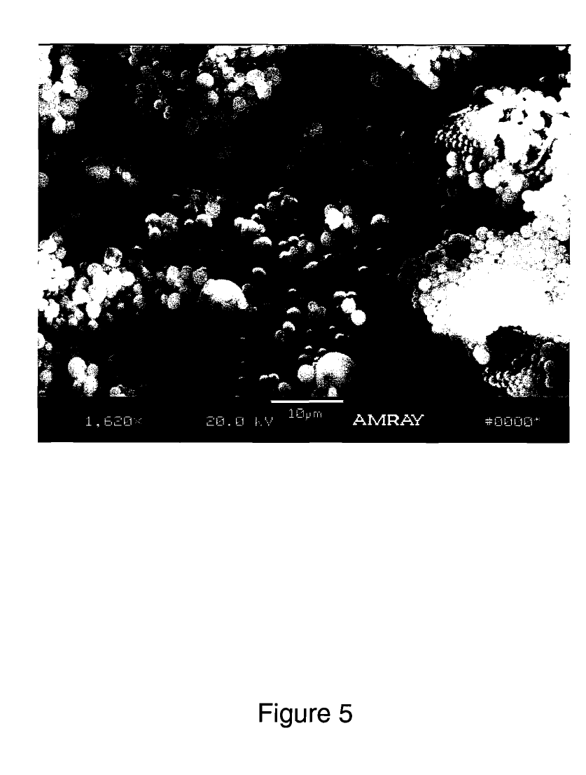

As observed in FIG. 3 through FIG. 5, the increase of methylene chloride yielded capsules that had the desired morphology. Increasing the organic solvent, increases the organic/aqueous phase ratio affecting the overall emulsion viscosity. The inner aqueous phase forms the empty void within the bubble after sublimation of the ammonium carbonate. Increasing this phase volume can present a condition for a greater degree of aggregation of the inner droplets contained within the bubble (Cui et al, 2005, J. Biomed. Mater. Res. B. Appl. Biomater. 73:171-17). At lower organic phase volumes, i.e. increase in aqueous/organic ratio, a shorter time was also observed for the evaporation/hardening step of the fabrication. This rapid hardening results in insufficient time for the polymer and aqueous phase to separate. Along with the greater degree of aggregation within the bubble, this faster time can tend to cause precipitation of the organic phase to occur more rapidly, thus the honeycomb like indented surface at lower methylene chloride concentrations (Cui et al, 2005, J. Biomed. Mater. Res. B. Appl. Biomater. 73:171-17). Thus, increasing the organic phase decreases aqueous to organic phase ratio. This, in turn, has an effect on the emulsion droplet, allowing each phase to separate from each other properly creating the smoother surface. It is therefore beneficial for targeting purposes to increase the organic phase in preparation of PLA-COOH microcapsules.

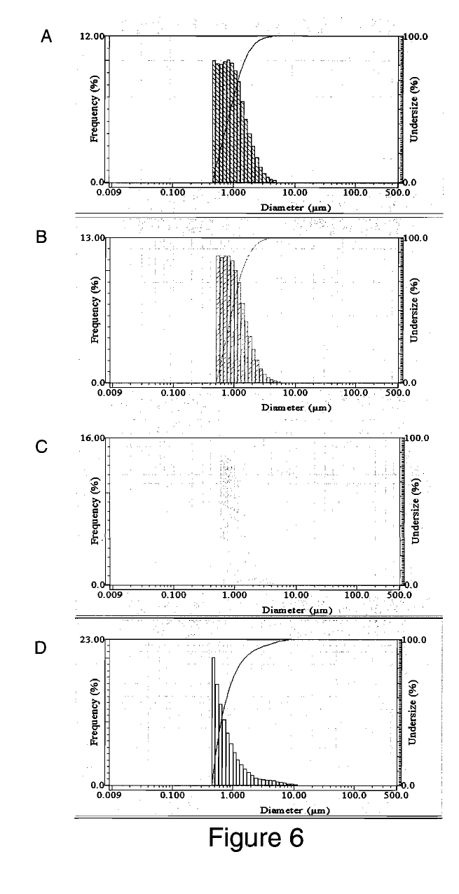

To determine the mean particle size of the microcapsules a Horbia instrument was used. FIG. 6 shows the size distribution.

b. Effect of Increasing Outer Organic Phase on In Vitro Dose-Response

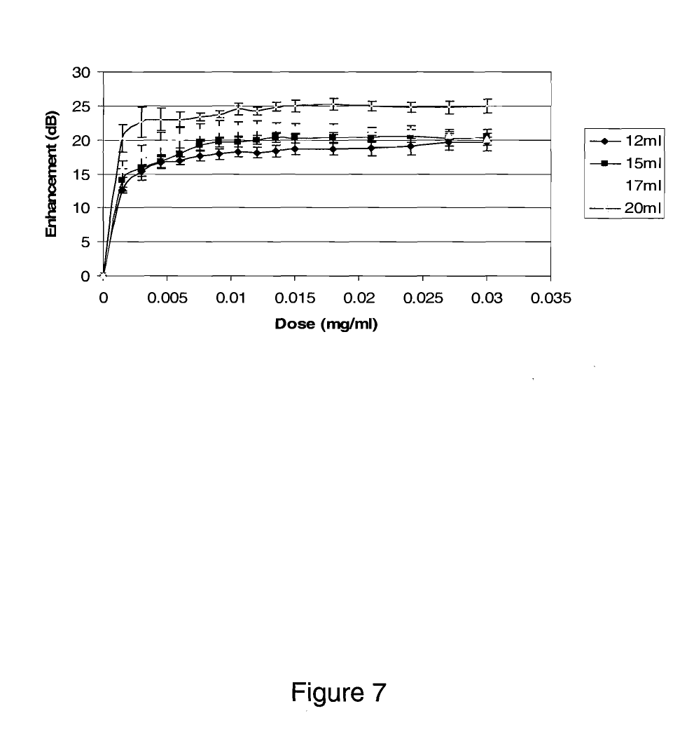

To determine the acoustic effects that the increased organic phase has, in vitro dose response studies were performed. All studies were performed at 37° C. in PBS (pH 7.4) to mimic in vivo characteristics. Dose response curves are shown in FIG. 7 for each modification of outer organic phase.

There is an increase in enhancement when the volume of methylene chloride increases, concurrent with the improvement in the shape of the capsules. A maximum enhancement (˜25 dB) was seen in the sample prepared with 20 ml methylene chloride at a dose of 0.018 mg/ml. Results from FIG. 7 were shown to be statistically significant in terms of the difference between the four groups by a one-way fixed ANOVA. Furthermore, post hoc comparison (Newman-Keuls) showed that samples made with 12 ml, 15 ml, and 17 ml methylene chloride were not statistically different with an α>0.05. The post hoc comparison though did show that these samples were significantly different from samples made with 20 ml methylene chloride with an α<0.05. Statistical testing did show two distinct groups, illustrating a difference in echogenicity when increasing the organic volume to 20 ml methylene chloride. There is then a point when the volume of organic phase further increases the enhancement of signal.

The sample prepared with 17 ml methylene chloride showed a slight decrease as the microcapsule dose increased (0.0015-0.003 mg/ml). This is considered to be shadowing that is observed at high sample concentrations. Bubbles closer to the ultrasound source obscure the acoustic wave from other bubbles, decreasing the transmission of sound waves and hence reducing signal power. However, as the organic phase parameters were changed, the microcapsules still proved to be sufficiently echogenic to have potential in vivo.

c. Effect of Increasing Outer Organic Phase on In Vitro Time Response

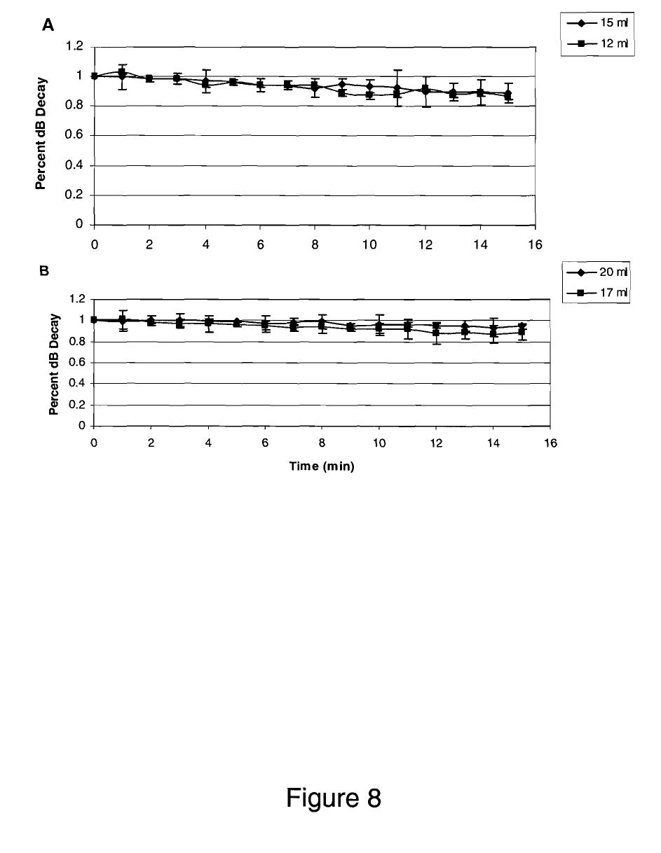

To investigate the CA's stability over time under constant insonation, in vitro acoustic enhancement studies were performed. Microcapsules were prepared of PLA-COOH with varying volumes of methylene chloride and tested at 37° C. over a period of 15 minutes. A dose was chosen depending on the dose response curve obtained. This was determined by examining the rise to the curve and selecting a dose near the saturation of enhancement. This allows for a more accurate measure of decreased echogenicity. Time decay curves were normalized to 1.0 to allow for comparison as shown in FIG. 8.

Each time response shows stability over the 15 minute period, losing about 10%-15% signal. Statistical analysis (Newman Keuls) showed that the samples prepared with 12 ml, 15 ml, and 17 ml were not statistically significant from one another at a α>0.05. The post-hoc comparisons did show that the samples prepared with 20 ml were statistically significant from the samples prepared with 12 ml, 15 ml, and 17 ml at a α<0.05. Similar to the previous dose response results, the samples prepared with 20 ml methylene chloride show a significant difference compared to other samples. These results suggest that the organic phase volume influences the performance of the CA when using non end capped PLA, and should be considered in the design of a targeted ultrasound CA.

d. Process Effects on PLA Molecular Weight

To further investigate the influences of preparation parameters in the fabrication of a CA, GPC analysis was performed (data not shown). The molecular weight of PLA was determined before and after the double emulsion method. The separation is based on differences in molecular size in the solution compared to standards that are used to determine the molecular weight of species eluting from the column. According to GPC results, it was not possible to distinguish the molecular weight differences quantitatively. The wide peaks seen indicate that the column used could have been functioning incorrectly to elute the specific sizes (M_{w}) of the polymer, or that there was a broad molecular weight range present in all samples, which did not vary significantly from sample to sample. However it can be concluded qualitatively that the M_{w} before and after the double emulsion process (factoring each parameter individually), is very similar. The PLA control (before fabrication) overlaps with each varied experimental factor, indicating that there is virtually no detectable change in the M_{w} of PLA.

e. In Vivo Tumor Imaging Using Microcapsules

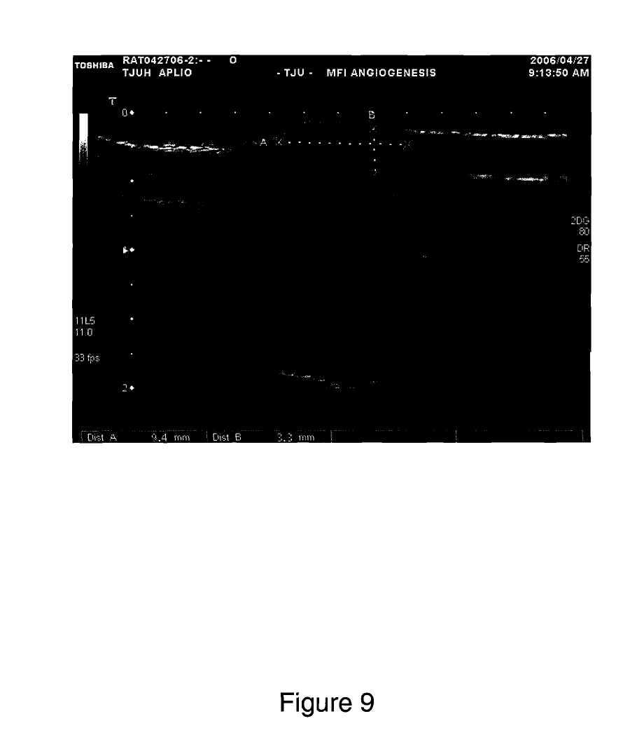

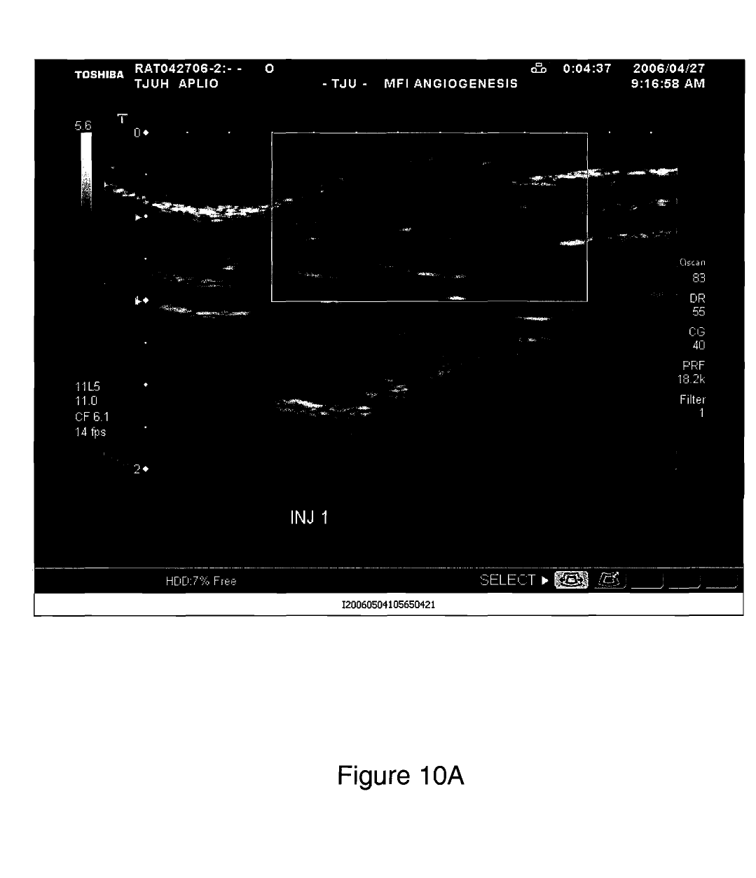

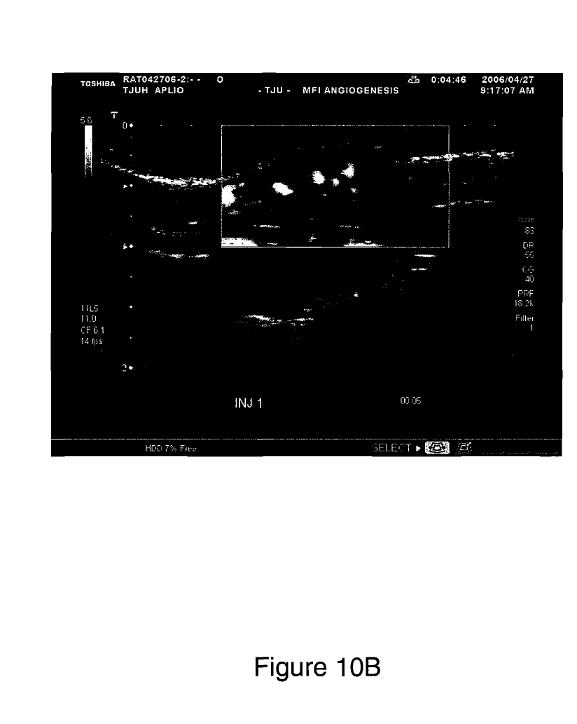

To evaluate the potential effect of using PLA-COOH as an ultrasound CA, an in vivo experiment was performed as a part of a larger study our lab conducted. The PLA-COOH CA was evaluated on its ability to image the developing microvessels associated with angiogenesis in an in vivo rat tumor model. A Sprague-Dawley rat was injected in the hind fat pad with NMU-induced cancer cells (1.5 million) and a sufficient tumor developed in about 7 weeks. The blood flow around and into the tumor was evaluated pre and post injection of the PLA-COOH CA with grey scale and power Doppler imaging as seen in FIG. 9 through FIG. 10A and FIG. 10B. PLA-COOH prepared with 20 ml methylene chloride was chosen due to it showing the highest acoustic enhancement in vitro.

The PLA-COOH CA illustrates a qualitative difference between pre and post injection. As seen in the Power Doppler image, the CA enhances and demonstrates the ability to show blood flow in the vasculature and in the tumor.

### Example 2

### Development of PLA Nanocapsule

The purpose of scaling down from microcapsules to nanocapsules is for use in targeted therapeutic imaging and drug delivery applications. A novel approach was undertaken to develop a nanosize ultrasound CA. In order to test the new method, solid capsules were first made and poly (DL-lactic acid) with an ester end cap was chosen based on the previous studies with PLA-COOH. Due to conflicting literature in discussing the influences of process parameters, the salting out method used to produce particles was investigated to determine the specific influences of experimental parameters in achieving the desired size. Numerous factors such as aqueous/organic phase ratio, PVA concentration and molecular weight, PLA concentration, and stirring speed were individually varied to observe the affect on capsule size.

a. Solid Poly(Lactic Acid) Nanoparticle—Variation in Process Parameters

The overall desire is to produce an ultrasound CA that has a nanometer size range. The investigation to produce particles of that preferred size was initiated using a salting-out method in which a water-miscible organic solvent (i.e. acetone) is emulsified in an aqueous phase saturated with salt. An important step in preparing the emulsion is the droplet size that eventually determines the final mean size of the particles. Parameters of the organic and aqueous phases that alter viscosity such as PVA concentration, molecular weight, and PLA concentration were varied to determine a basis to further explore the formulation of echogenic particles.

### 1. PVA Percentage

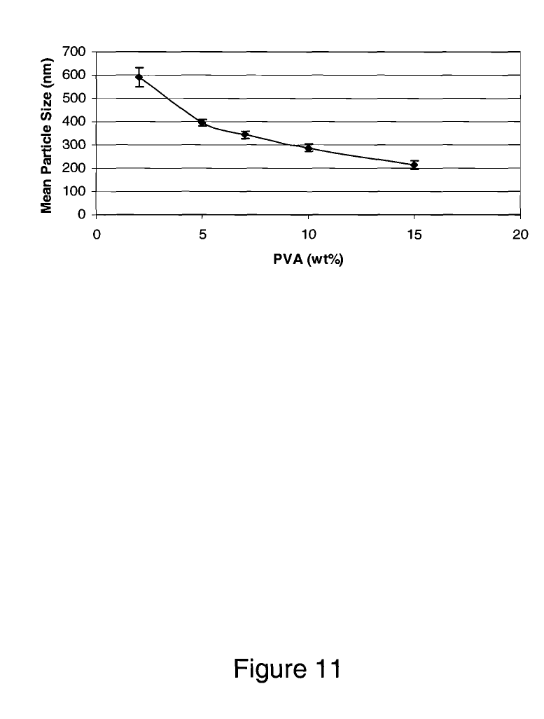

Viscosity of the emulsion can change significantly due to the concentration of poly(vinyl alcohol) (PVA) in the aqueous phase. This viscosity of the aqueous phase has been shown to have an important role in determining particle size. Murakami et al showed an increase in diameter size, relating it to an increase in viscosity of the emulsion (Murakami et al., 1997, Intrnl. J. Pharmaceutics 149:43-49). In contrast, Alleman et al. (1992, Internl. J. Pharmaceutics 87:247-253) reported a decrease in size with increasing viscosity relating it to a steric stabilization. Therefore, increasing PVA concentrations were investigated from 2 wt %-15 wt %. Particle size decreased with increasing PVA concentrations as illustrated in FIG. 11 and shown in Table 2.

TABLE 2
Influence of percentage of PVA on the particle size.

| | | Polydispersity |
|---|---|---|
| PVA Concentration | Mean Particle Size (nm) | Index ± Standard |
| (wt %) | Standard Deviation | Deviation |
| 2 | 590.6 ± 56.8 | 0.645 ± 0.34 |
| 5 | 385.5 ± 25.1 | 0.367 ± 0.14 |
| 7 | 343.7 ± 14.6 | 0.239 ± 0.12 |
| 10 | 288.2 ± 15.9 | 0.112 ± 0.03 |
| 15 | 213.7 ± 11.3 | 0.092 ± 0.04 |

Polymer concentration (5.0 wt %), PVA(25 kDa), 2.5 aqueous/organic phase ratio, stirring speed (2000 rpm) held constant.

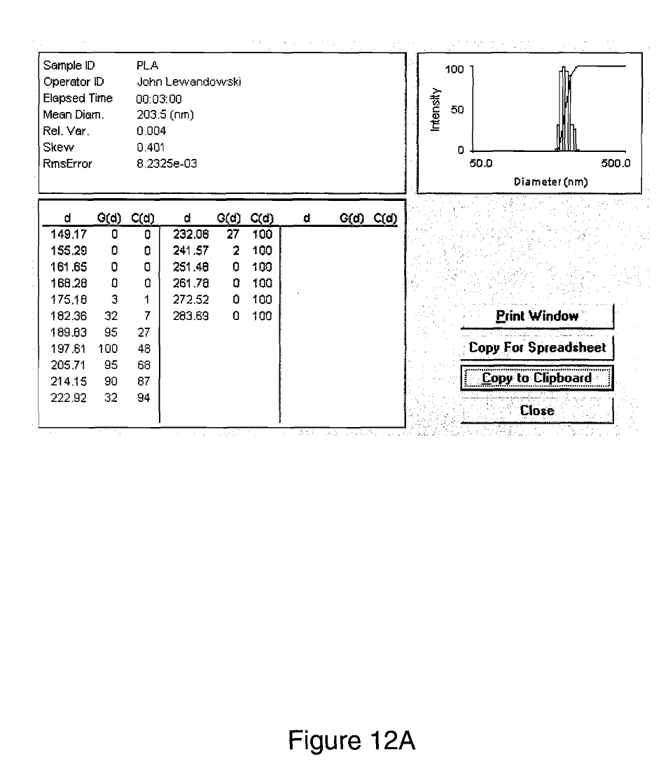

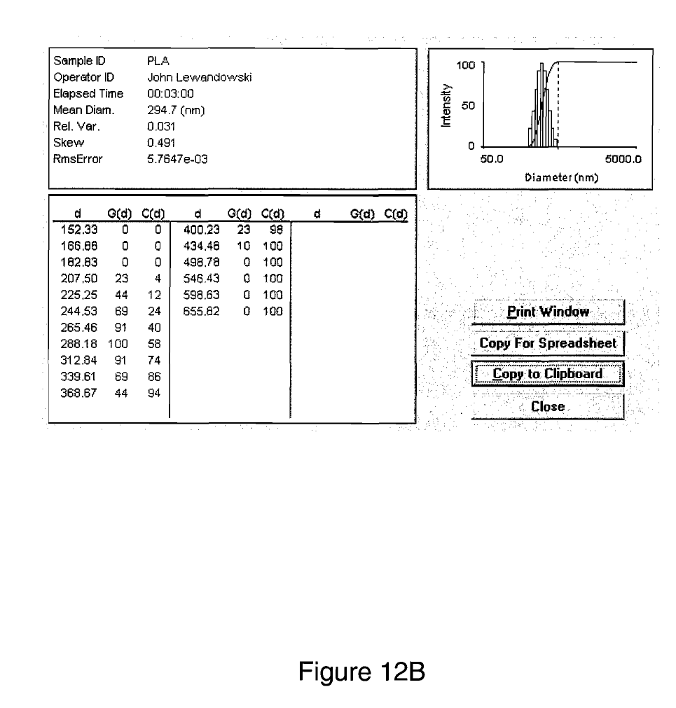

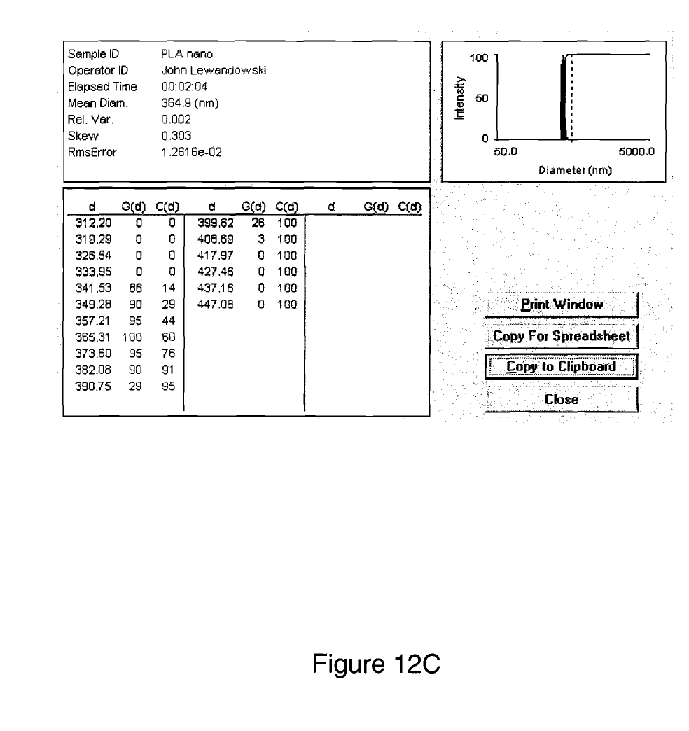

Nanoparticles created also showed distributions that are characteristics of being monodispersed as shown in FIG. 12. A dynamic light scattering, particle size analyzer used due to the cut-offs the Horbia displayed.

As shown, increasing the concentration of PVA decreases the particle size ranging from 659.3 nm-189.4 nm. An upper limit of concentration is reached at 15 wt % PVA, at which point the aqueous gel in preparation becomes very viscous, almost to the point that it gets very difficult to produce the aqueous phase. There is also a limitation on how high the PVA concentration can be due to the constant 60 wt % MgCl₂*6H₂O. With the increase of PVA concentration in the aqueous phase, the water weight percentage as a result decreases to the point were the aqueous phase becomes difficult to prepare.

The particle size is dependent on the raw droplet size formed during the emulsion stage. It is possible that the decrease in size can be attributed to the polymer chains of the PVA interacting at the emulsion droplet surface. As PVA increases to the point where there is a sufficient amount, the particle size becomes steady resulting in uniform size particles (Galindo-Rodriguez et al., 2004, Res 21:1428-1439). The size and polydispersity index are much broader at lower PVA concentrations.

As discussed above, there is conflicting literature to determine the trend involved with PVA concentration. It should be pointed out that many of these studies involve different preparation methods and the use of different solvents such as acetone in comparison to methylene chloride. Niwa et al. (1993, Journal of Controlled Release 25:89-98), using a solvent diffusion, reported a significant decrease in size to submicron level when acetone was employed as the organic solvent, in comparison to methlyene chloride, and showed it as a decrease in interfacial tension and the solubility of the solvent (Niwa et al., 1993, Journal of Controlled Release 25:89-98). As mentioned earlier, there is a point at which the mixture becomes more viscous contributing to smaller efficiency of emulsification though at the same moment an increased stabilization.

### 2. PVA Molecular Weight

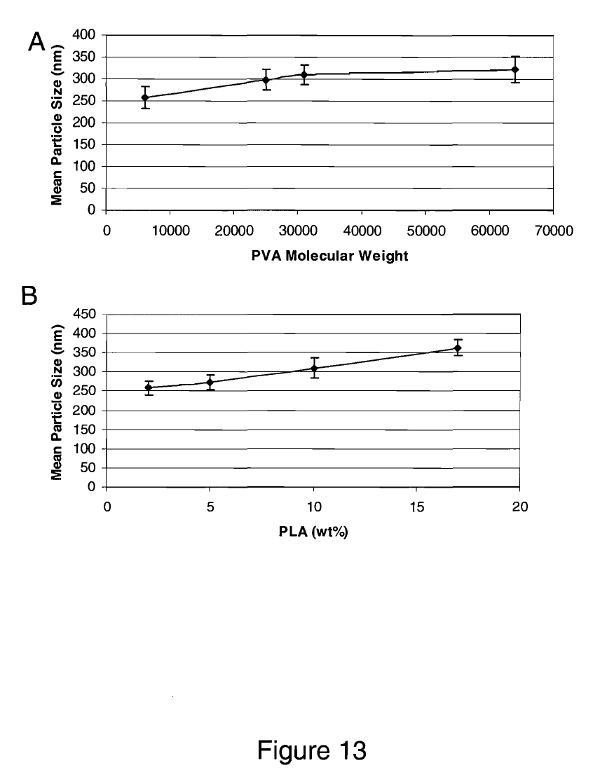

The PVA molecular weight in the preparation was investigated to observe potential effects on the particle size. Molecular weight has the ability to affect the viscosity of the emulsion and hence the final particle size. PVA molecular weight was varied with a constant hydrolysis of 88% to determine effect on size as shown in FIG. 13A. PVA is prepared by hydrolysis of poly vinyl acetate to remove acetate groups. Therefore percent hydrolysis refers to the amount of poly vinyl acetate that is hydrolyzed and the number of hydroxyl groups on the surfactant (Murakami et al., 1997, International Journal of Pharmaceutics 149:43-49). PVA concentration of 10 wt % was chosen based on the previous size results where the preparation of aqueous phase was still manageable.

The increase in PVA molecular weight showed a slight increase in the particle size agreeing with the conflicting literature. During the procedure from observation, the viscosity did increase slightly with increasing molecular weight that could have an effect on the mixing efficiency. Scholes et al., 1993, Journal of Controlled Release, 25:145-153 reported a similar effect producing PLGA nanoparticles. They found an increase in particle size with increasing viscosities that was linked to poorer mixing (Scholes et al., 1993, Journal of Controlled Release, 25:145-153). Results though from FIG. 13 were shown not to be statistically significant in terms of the difference between the four groups by a one-way fixed ANOVA (α>0.05). These statistical results show that the different PVA molecular weights did not have a considerable effect on the mean particle size.

### 3. PLA Percentage

The PLA percentage in the organic phase in the preparation was also investigated to observe potential effects on the particle size. As with PVA molecular weight, PLA concentration has the ability to affect the viscosity of the emulsion and hence to influence the final particle size. PLA percentage was varied from 2 wt %-17 wt % and the resulting effect on particle size is shown in FIG. 13B.

A small increase in particle size was seen when polymer concentrations in the organic phase increased. It was also noted that the samples only yielded about 50% of the initial PLA weight used (data not shown) so at 2 wt % the amounts were very sparse. Results from FIG. 13A were shown to be statistically significant in terms of the difference between the four groups by a one-way fixed ANOVA. Furthermore, post hoc comparison (Newman-Keuls) showed that samples prepared with 2, 5, and 10 wt % PLA were not significantly different from each other (α>0.05). Though, it was also shown that this group was significantly different (α<0.05) then samples prepared with 17 wt %. These statistical results show two separate groups that can possibly be explained by an increase in viscosity of the emulsion producing higher shear forces in mixing (Kwon et al., 2001, Physicochemical and engineering aspects 182:123-130). There is then a limit when the viscosity of the emulsion is affected enough to have an influence on particle size.

b. Morphology of Nanoparticles

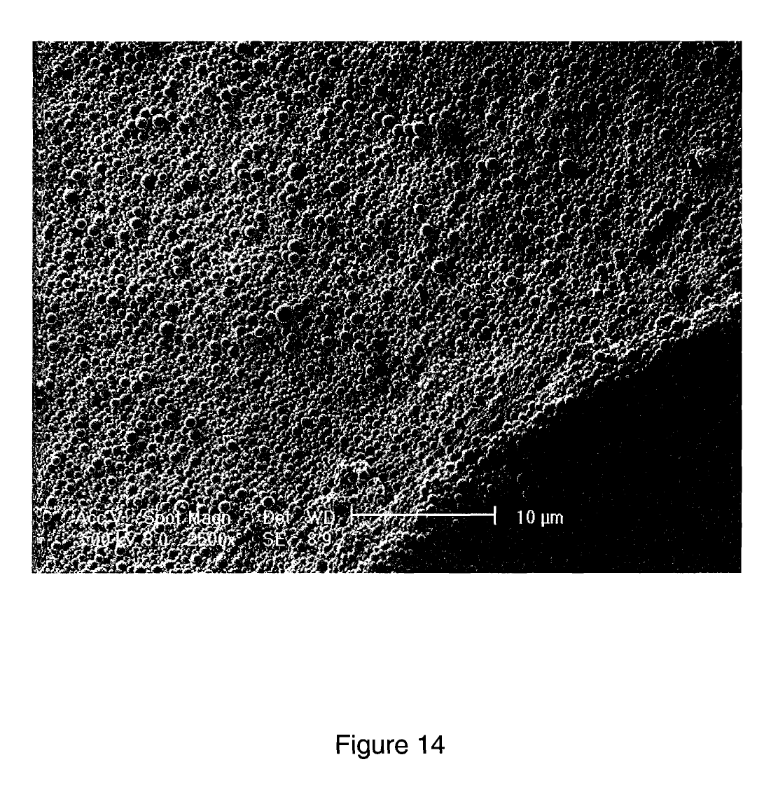

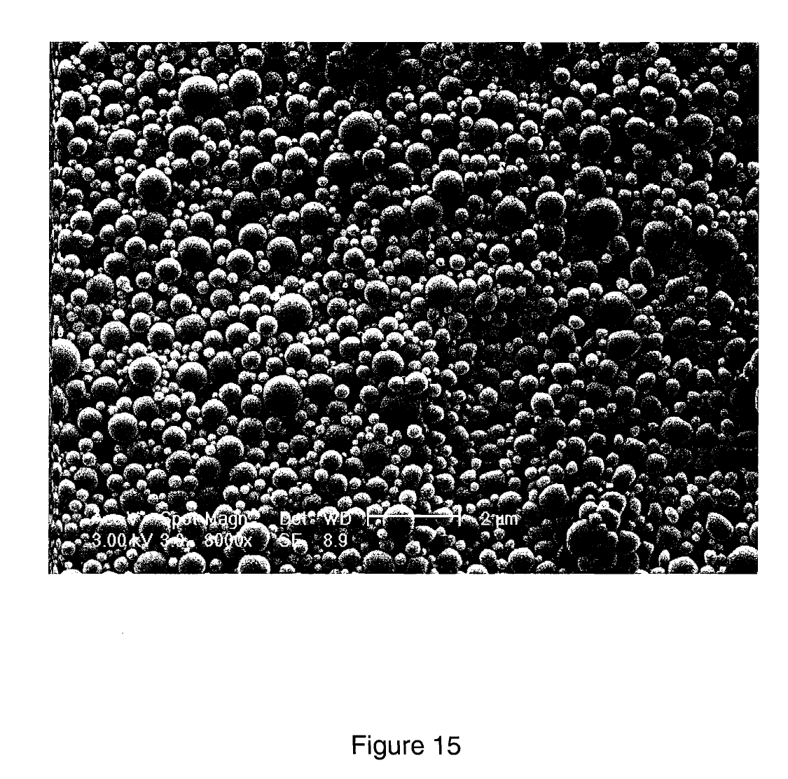

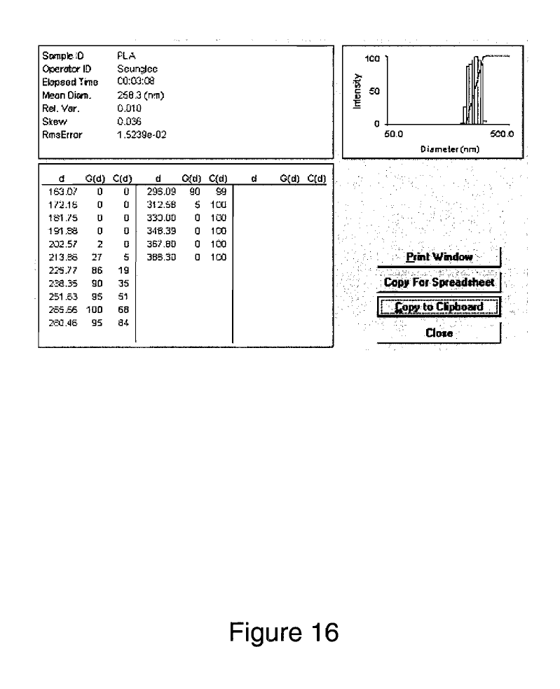

To examine the surface morphology of the nanoparticles, an SEM was used. This was employed to investigate the shape and verify the size of the particles. The optimized particles determined from the previously described studies were imaged. High and low magnification SEM pictures can be seen in FIG. 14 and FIG. 15. As seen in the SEM figures, about 80-90% of the particles are between about 50-500 nm with very few around 800 nm-1 um. Size analysis revealed a mean particle size around 260 nm (FIG. 16).

### Example 4

### Development of and Ultrasound Contrast Agent

A further aspect of this work is to investigate the possibility of developing a nanosize ultrasound CA using the salting out method. In creating a CA, there must be an impedance mismatch to reflect the ultrasound signal. To accomplish this, a sublimable porogen is added to both the organic and aqueous phases and later removed through lyophilization leaving a void. Therefore, the potential of a porogen core within the nanoparticle was investigated in the salting out procedure. Moving from a microcapsule to a nanocapsule will be used in future targeted therapeutic imaging and drug delivery applications. Such applications include blood vessels found in tumors which show leakiness due to the presence of open gaps at some endothelial junctions. Transport can occur through these openings, which have been reported to range in size between 380 and 780 nm for several tumor models (Moghimi et al., 2001, Pharmacol Rev 53:283-318). Modifying the salting out procedure, a capsule was fabricated that displayed echogenicity. External (PVA) and internal phase (PLA) parameters of the method were investigated to determine affect on size, stability, longevity, and echogenicity of the capsule.

Expanding on the results and studies performed to produce solid nanoparticles, certain parameters were chosen to create an echogenic nanocapsule. PVA with a molecular weight of 25 kDa was chosen based on previous size and stability results. It was shown that PVA with this M_{w} has the ability to stabilize the emulsion and still have the ability to produce solid nanoparticles at a mean size of about 298 nm. The higher molecular weight leads to a more stable particle which is directly related to the surfactant strength.

The concentration of PVA and PLA concentration previously showed to have a role in determining the final capsule size in the salting out procedure but its effect on the capsule's ability to reflect ultrasound has not been investigated. Therefore these two parameters of the aqueous and organic phase, respectively, were examined. A mixing speed of 2000 rpm and aqueous/organic phase of 2.5 was held constant as in previous studies to produce solid nanoparticles. A concentration of 1.0M ammonium carbonate in inner aqueous phase and camphor (10 wt % of the PLA) was used as the porogen based on previous studies of fabricating a PLA endcapped CA.

a. Influence of PVA Concentration in Aqueous Phase

### 1. Size Analysis

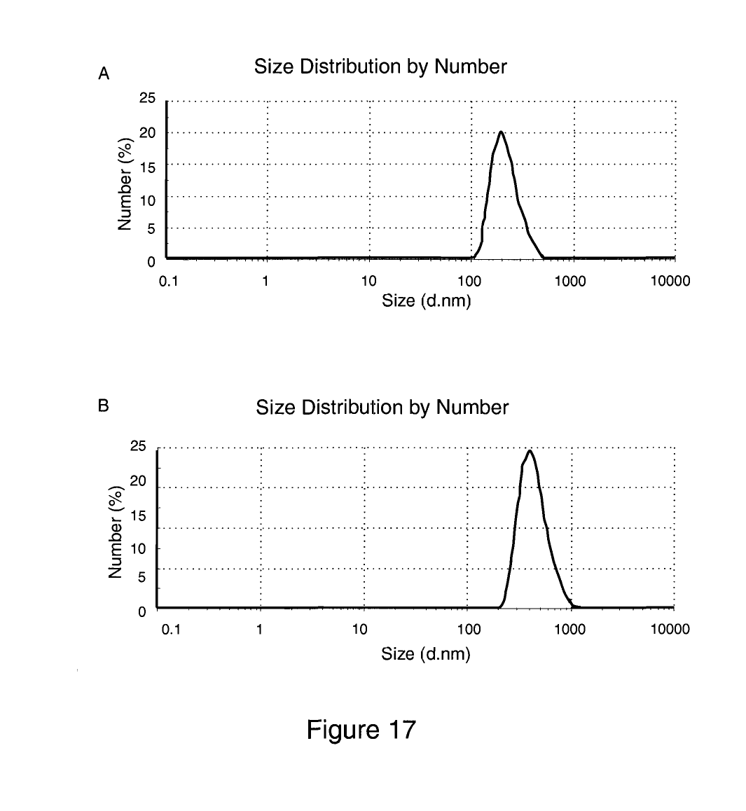

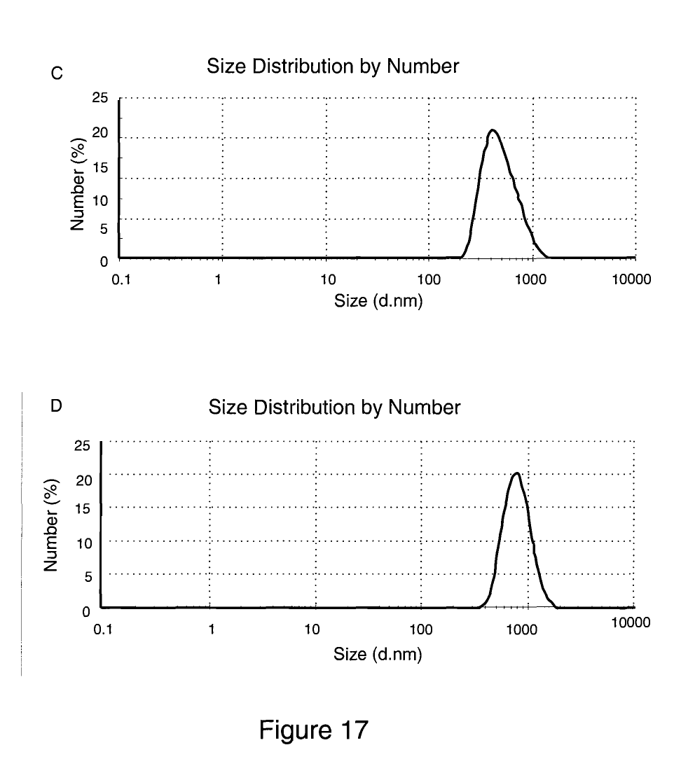

In modifying the salting out procedure to produce a CA, the effect of PVA concentration on the size of the echogenic particle was examined. PLA concentration was held constant (5 wt %) due to the previous results to produce the desired yield and size of solid nanoparticles (Table 3 and FIG. 17). Particle size analysis was performed by a Malvern Zeta Sizer (nano series).

TABLE 3
Influence of PVA concentration on mean particle size.

| | Mean Capsule Size | |
|---|---|---|
| PVA Concentration | (nm) ± Standard | Polydispersity |
| (wt %) | Deviation | Index |
| 2 | 817.2 ± 37.2 | 0.459 ± 0.010 |
| 5 | 640.0 ± 18.4 | 0.308 ± 0.027 |
| 10 | 486.2 ± 9.5 | 0.259 + 0.016 |
| 15 | 261.3 ± 17.3 | 0.123 ± 0.017 |

Polymer concentration (5.0 wt %), PVA (25 kDa), 0.04 g camphor, 1M Ammonium Carbonate, 2.5 aqueous/organic phase ratio, stirring speed (2000 rpm) held constant.

Increasing the PVA concentration showed a similar trend as was observed in producing solid nanoparticles. The size of the particle decreased as the PVA concentration increased as shown in Table 3 and the shift of the size distributions is shown in FIG. 17.

### 2. In Vitro Dose Response

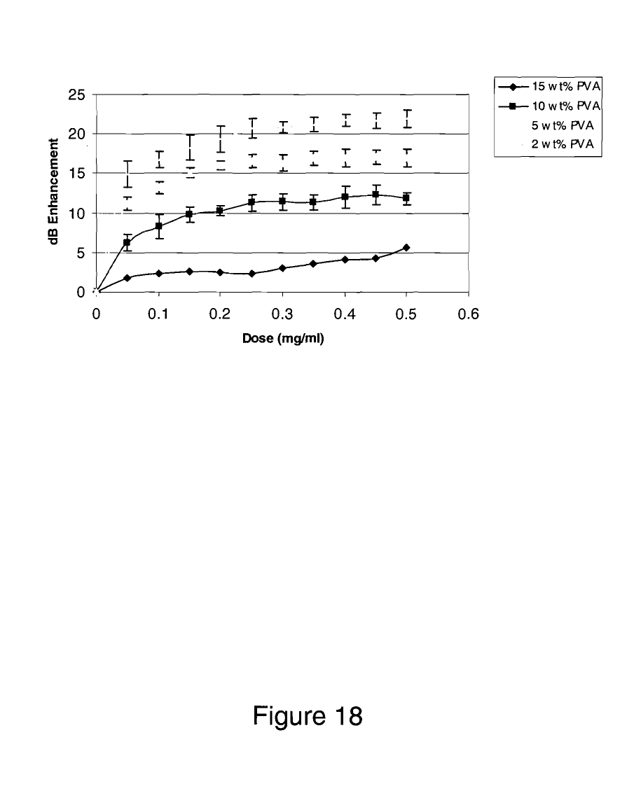

To evaluate this method to produce an ultrasound CA, a cumulative dose response curve was constructed to assess the capsules' acoustic properties. As shown in FIG. 18, while maintaining a constant PLA percentage, increasing the amount of PVA (2 wt %, 5 wt %, 10 wt %, 15 wt %) in the aqueous phase resulted in a decrease in dB enhancement.

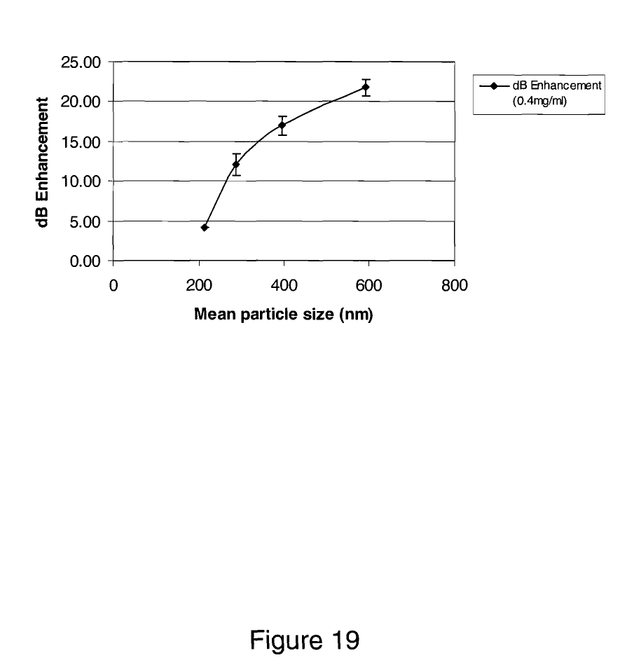

A maximum enhancement was seen with the 2 wt % PVA around 22 dB at a dose of 0.4 mg/ml while 15 wt % PVA exhibited minimal enhancement (˜5 dB). It should be noted that the dose of the particles required to achieve maximum echogenicity increased significantly (0.4 mg/ml vs. 0.04 mg/ml) in comparison to the microbubbles. Using sample concentrations that are similar in testing microbubbles (stock solution of 15 mg/ml), the results produced little to no enhancement (data not shown). FIG. 19 illustrates the dB enhancement as the mean size of the particle increases.

As shown in FIG. 19, the echogenicity (at 0.4 mg/ml) increases as the mean particle size increases. By varying the PVA concentrations in the aqueous phase, the mean diameter as well as the echogencity can be altered. The amount of PVA determines the size but as well determines the stability and viscosity of the emulsion. As the PVA concentrations in the aqueous phase decreased, the size increased as well and the ability to control the size distribution. When PVA concentrations increased and the emulsion also became significantly viscous causing a negative effect on producing echogenic particles.

Given that the resonance frequency is dependent of CA diameter (f₀˜6500/d), it would be hypothesized that the resonance frequency of the nanocapsules would be much greater than the microcapsules and thus a dose response at 5 MHz would not show much acoustic enhancement. Therefore, the increased concentration that is needed for acoustic enhancement must be taken into account. However, it is also possible that nanoparticles contain a small population of microparticles that are contributing to the echogencity and an increase in concentration is therefore needed to achieve an adequate concentration of these larger capsules. It is of significance to note that to date of writing this work, there is no knowledge of the fabrication of an ultrasound CA via the salting out procedure. This information is important for the further development and optimization of echogenic capsules that could be used for ultrasound imaging.

### 3. In Vitro Time Response

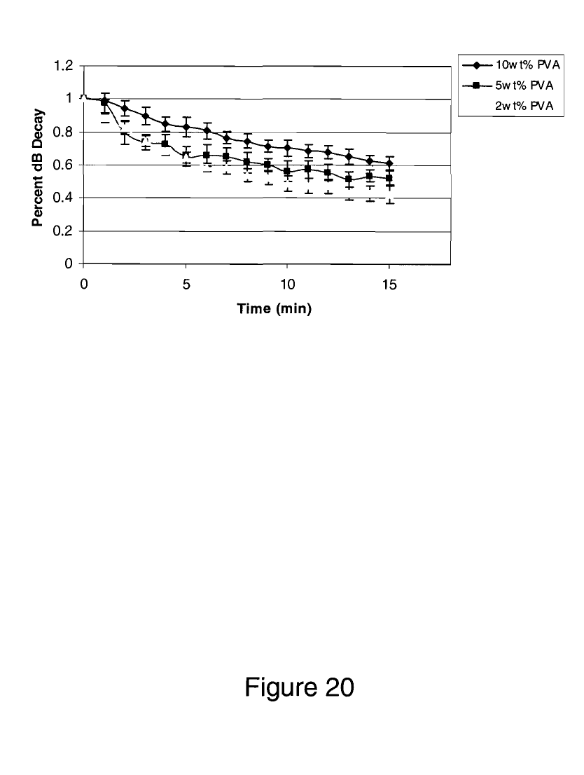

To determine the stability of the CAs, the acoustic response under constant insonation was measured over time. FIG. 20 shows the enhancement decay over a period of 15 minutes.

After 15 minutes of insonation by ultrasound, the sample still displayed echogencity. As shown, increasing the concentration of PVA, which plays a role in the shell strength, decreases the signal loss. Samples prepared with 10 wt % PVA showed the smallest decay, loosing about 40% of signal total in comparison to 60% total loss of signal exhibited by the 2 wt % PVA samples. One way fixed ANOVA analysis did show a significant difference among the groups. Further, statistical analysis (Newman Keuls) showed samples prepared with 2 wt % and 5 wt % PVA where not significantly different (α>0.05). It did show a significant difference among the group of samples prepared with 2 wt % and 5 wt % in comparison to the 10 wt % PVA (α<0.05). These results suggest the percentage of PVA used to produce the capsules, affects the stability of the bubble. There is a point where the increased concentration of PVA acts to increase the strength of the capsule's shell, decreasing signal decay over time. This reduction in acoustic enhancement at 37° C. may be due to several conditions. A CA uses a surfactant such as PVA to stabilize the shell. The salting out procedure also makes use of PVA but more as a stabilizing colloid then a surfactant. Additionally, in comparison to the double emulsion method, an evaporation phase to remove the solvent uses isopropyl alcohol and hexane to harden the shell of the microcapsule where in the salting out procedure, this is not performed since salt hydration causes polymer precipitation. Therefore it is hypothesized that the shell thickness is smaller when using salting out and hence weaker when exposed to ultrasound energy. However, these results do tend to indicate that the echogenicity is not a result of a few large capsules, since they would all be expected to have similar degradation half lives, not be a function of mean size.

b. Influence of PLA Concentration in Organic Phase

Viscosity of the emulsion has been shown to have an effect on size and echogencity of the resulting particle. PLA concentration in the organic phase can contribute to this viscosity thus the influence of PLA concentration on echogencity was investigated. PVA was held constant at 5 wt % based on the previous dose response curve and smaller size analysis in comparison to 2 wt %. A long term goal is to evaluate the in vivo enhancement. Therefore, enhancement in vitro must be sufficient enough to be have an effect in the physiological environment.

### 1. Size Analysis

As shown in Table 4, there is no trend seen in particle size when increasing the polymer concentration in the organic phase. This is similar to the observed results when fabricating solid nanoparticles using the salting out method at the same PLA concentrations.

TABLE 4
Influence of polymer concentration on mean particle size.

| | Mean Particle Size (nm) ± Standard |
|---|---|
| PLA Concentration (wt %) | Deviation |
| 2 | 611.2 ± 23.8 |
| 5 | 640.0 ± 18.4 |
| 10 | 632.8 ± 17.9 |

PVA (25 kDa) concentration (5.0 wt %), 0.04 g camphor, 1M Ammonium Carbonate, 2.5 aqueous/organic phase ratio, stirring speed (2000 rpm) held constant.

### 2. In Vitro Dose Response

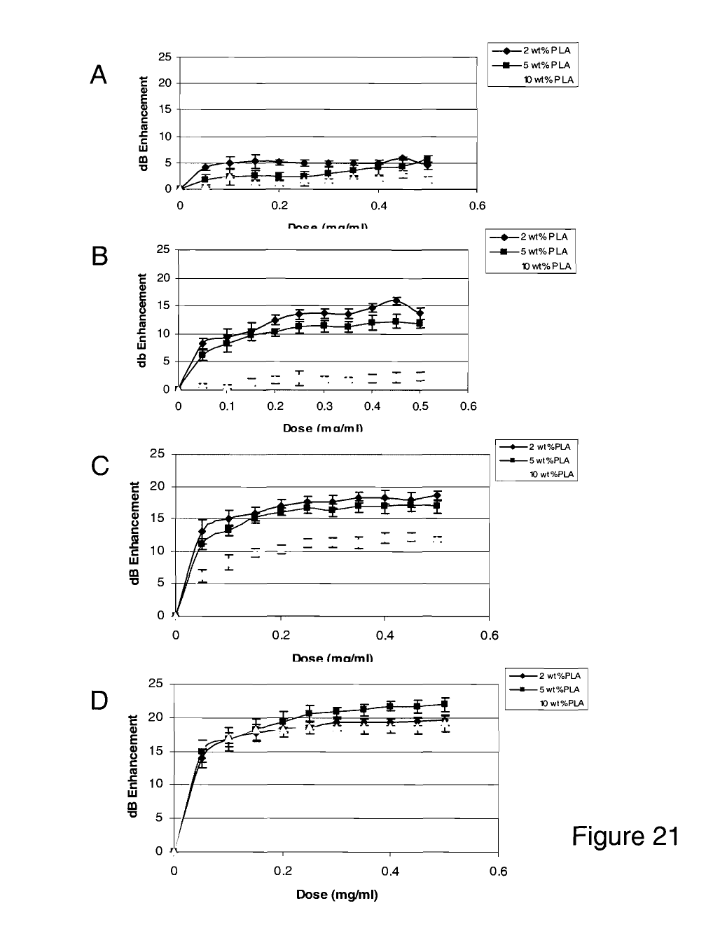

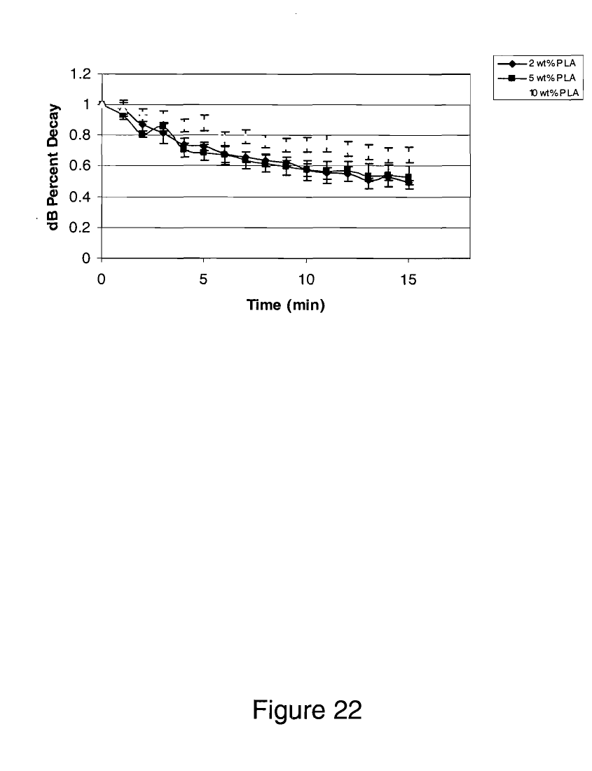

The viscosity of the emulsion determines the ability to produce echogenic particles. Therefore a parameter of the organic phase, PLA percentage, was varied to determine the effect on dB enhancement at different PVA concentrations. A cumulative dose response curve was constructed to assess the capsules acoustic properties where PLA concentrations were varied from 2 wt % to 5 wt % to 10 wt %, with PVA (25 kDa) concentrations of 15 wt %, 10 wt %, 5 wt %, and 2 wt % as shown in FIG. 21.

As shown in FIG. 21, a similar trend is observed in comparison to previous results when PVA percentage is increased. Fabricating samples with a PVA concentration at an upper limit of 15 wt %, produced very little enhancement, independent of PLA percentage. However, increasing the PLA to 10 wt %, the viscosity was modified enough to have an influence on the echogencity of other samples. This trend was observed in samples prepared with PVA of both 5 wt % and 10 wt % but was absent with 2 wt %. At this low concentration of 2 wt % PVA, it is possible that the emulsion viscosity is not affected enough to influence echogencity by the amount of PLA in the organic phase. Adjusting the polymer concentration higher then 10 wt % was not investigated, but it is presumed that there is a significant point (dependent as well on PVA concentration) where the percentage of PLA effects the viscosity of the emulsion. A maximum signal enhancement was shown at lower concentrations of polymer (2 wt %) of 15, 18 and, 21 dB among samples prepared with PVA of 10, 5, and 2 wt % respectively. The results show the PLA percentage in the organic phase has an effect on the echogencity of the capsule.

### 3. In Vitro Time Response

To determine the stability of the CAs, the acoustic response under constant insonation was measured over time. FIG. 20 shows the enhancement decay over a period of 15 minutes.

After 15 minutes of insonation by ultrasound, the samples prepared with 5 wt % PVA still displayed echogencity similar to the previous results. Samples prepared with 10 wt % PLA showed the smallest decay, loosing about 35% of signal total in comparison to 50% total loss of signal exhibited by the 2 wt % PLA samples. One way fixed ANOVA analysis did show a significant difference among the 3 groups. Further, statistical analysis (Newman Keuls) showed samples prepared with 2 wt % and 5 wt % PLA where not significantly different (α>0.05). It did show a significant difference among the group of samples prepared with 2 wt % and 5 wt % in comparison to the 10 wt % PLA (α<0.05). The increased PLA concentration in the organic phase therefore enhances the stability of the capsule but also shows to decrease the signal enhancement shown in the dose response curves.

c. Morphology of Salting Out Contrast Agents

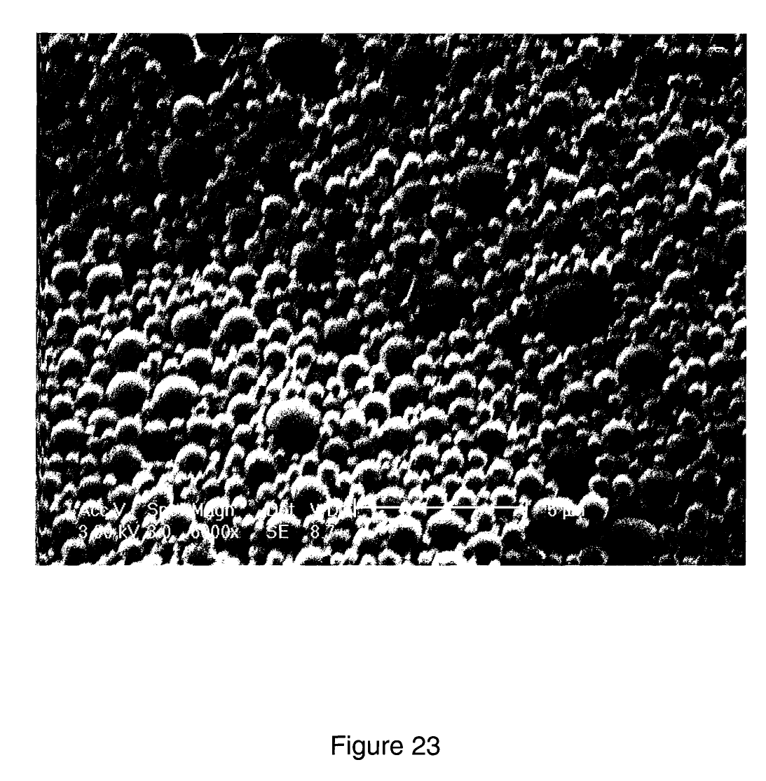

To examine the surface morphology of the nanoparticles, a SEM was used. This was employed to investigate the shape and observe the size of the particles (FIG. 23).

As seen in FIG. 23, the particles are spherical in shape and size analysis revealed a mean particle size around 640 nm (FIG. 16 and Table 3). There is a small population of larger bubbles present as well.

Utilizing the salting out method presents many advantages to substitute one experimental factor for another to further control the resulting particle. The present invention contemplates the use of other organic solvents, salting out agents, polymers such as PLGA, variable M_{w} of PLA, and stirring blade in the fabrication process as well as selection processes, such as size exclusion or filtration to obtain a population of nanoparticles enriched for a specific size range.

### Example 5

### Drug Loaded Contrast Agent

Camphor (0.04 g) and poly lactic acid (7.56 g) are dissolved in 7.6 g of acetone by stirring for 40 minutes. While the organic phase is being prepared, 8 mg of doxorubicin (2% by wt of PLA) is dissolved in 1 ml of 1.0M ammonium carbonate solution in an eppendorf tube. The aqueous phase is prepared by first weighing a beaker before placing any materials inside. Next, water (35 g) and MgC₁₂*6H₂O (60 g) are combine in the beaker. A magnetic stir bar is added and the beaker is placed on hot/stir plate. The salt is dissolved in the water while increasing the temperature of the solution to about 80° C. PVA (5 g) is added slowly (˜0.1 g increments) to the beaker when it reaches 80° C., stirring constantly to prevent clumping. The beaker is stirred for about 3 hours at constant temperature, adding water to adjust for evaporation. After 3 hours, the beaker is cooled and the beaker re-weigh to confirm proper weight (˜100 g aqueous phase) or adjust it by adding water or continuing heating. When the aqueous phase is fully cooled, weigh out 20 g and place remainder aside for future use.

After the organic phase is completely dissolved, the previously prepared 1 ml solution of Ammonium Carbonate/Doxorubicin mixture is added to the organic phase. It is then mixed by pulse sonication at 110 W for 30 seconds at intervals of 3 seconds on and 1 second off creating a water-in-oil (W/O) phase. Next, the W/O phase in poured into a 250 ml beaker and 20 g of aqueous phase is added dropwise, under mechanical stirring of 2000 rpm. Stirring of the solution occurs until an O/W emulsion forms (˜10 min). Distilled water (50 ml) is then added to the emulsion while stirring is continued to cause the remaining acetone to diffuse into the aqueous phase. After addition of the distilled water stirring is continued for 30 seconds. The resulting solution is centrifuged three times at 16,000 rpm for 20 minutes (3×) to collect the capsules and purify by removal of excess salt, PVA, and organic solvent. The purified capsules are resuspended in distilled water in a wide necked tube, a Kim wipe is secured on top of the tube with a rubber band, and the contents is frozen at −80° C. for at least 30 min or until thoroughly frozen. The frozen sample is place onto a freeze dryer vessel and freeze dried for about 48 hours to remove the ammonium carbonate and camphor and residual water.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of making polymeric echogenic microcapsules and nanocapsules, the method comprising:
(1) emulsifying (e.g., mixing by sonication) an organic phase with a first aqueous phase to provide a first water in oil emulsion;
(2) sequentially adding a dose of a second aqueous phase to the first water in oil emulsion until an inversion oil in water emulsion is formed such that from 50 to 99% of a water miscible solvent from the organic phase is extracted from the organic phase into the second aqueous phase;
(3) adding water to the oil in water emulsion and thereby further extracting the water miscible solvent and forming polymeric microparticles and nanoparticles; and
(4) removing sublimable substances (e.g., by freeze drying) and thereby obtaining polymeric echogenic microcapsules and nanocapsules.

2. The method of claim 1, wherein the organic phase comprises a polymer and a non-water soluble sublimable substance which are dissolved in a water-miscible solvent.

3. The method of claim 1, wherein the first aqueous phase comprises a water soluble sublimable substance dissolved in water.

4. The method of claim 1, wherein the second aqueous phase comprises a salting-out agent (or a solvent extracting agent) and a stabilizing agent (colloid) dissolved in water, wherein the stabilizing agent is present in a highly concentrated solution of a salting-out agents or a solvent extracting agents in water.

5. The method of claim 1, wherein the polymer is poly(lactic acid), the non-water soluble sublimable substance is camphor, the water-miscible solvent is acetone, the water soluble sublimable substance is ammonium carbonate, the stabilizing agent is poly(vinyl)alcohol, and the salting out agent is magnesium chloride which is present in at least 50 wt % of the second aqueous phase.

6. A pharmaceutical composition comprising a nanosized contrast agent, wherein said contrast agent is manufactured by a method comprising the steps:
(1) emulsifying (e.g., mixing by sonication) an organic phase with a first aqueous phase to provide a first water in oil emulsion;
(2) sequentially adding a dose of a second aqueous phase to the first water in oil emulsion until an inversion oil in water emulsion is formed such that from 50 to 99% of a water miscible solvent from the organic phase is extracted from the organic phase into the second aqueous phase;
(3) adding water to the oil in water emulsion and thereby further extracting the water miscible solvent and forming polymeric microparticles and nanoparticles; and
(4) removing sublimable substances (e.g., by freeze drying) and thereby obtaining polymeric echogenic microcapsules and nanocapsules;

7. The pharmaceutical composition of claim 6, wherein said contrast agent further comprises a targeting moiety.

8. The pharmaceutical composition of claim 7, wherein said contrast agent further comprises a therapeutic agent.
